(19) United States
(12) Patent Application Publication
(10) Pub. No.: US 2009/0209561 A1
(43) Pub. Date: Aug. 20, 2009

(54) Xanthine Derivatives with HM74A Receptor Activity

(76) Inventors: Richard Jonathan Daniel Hatley, Hertfordshire (GB); Ivan Leo Pinto, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/577,763

(22) PCT Filed: Apr. 1, 2005
(86) PCT No.: PCT/US05/11376

§ 371 Date: Apr. 23, 2007

(30) Foreign Application Priority Data
Oct. 22, 2004 (GB) 0423568.5
Dec. 10, 2004 (GB) 0427079.9

(51) Int. Cl. A61K 31/522; C07D 473/04

(52) U.S. Cl. 514/263.22; 544/267; 514/263.34; 514/263.36; 544/269

(57) ABSTRACT

The present invention relates to therapeutically active xanthine compound derivatives, corresponding pharmaceutical formulations containing, processes for making or manufacture and the use of the aforementioned compounds in therapy, particularly in the treatment of diseases where under-activation of the HM74A receptor contributes to the disease or where activation of the receptor will be beneficial.

The present invention relates to therapeutically active compounds which are xanthine derivatives, processes for the manufacture of said derivatives, pharmaceutical formulations containing the active compounds and the use of the compounds in therapy, particularly in the treatment of diseases where under-activation of the HM74A receptor contributes to the disease or where activation of the receptor will be beneficial.

Dyslipidaemia is a general term used to describe individuals with aberrant lipoprotein profiles. Clinically, the main classes of compounds used for the treatment of patients with dyslipidaemia, and therefore at risk of cardiovascular disease are the stating, fibrates, bile-acid binding resins and nicotinic acid. Nicotinic acid (Niacin, a B vitamin) has been used clinically for over 40 years in patients with various forms of dyslipidaemia. The primary mode of action of nicotinic acid is via inhibition of hormone-sensitive triglyceride lipase (HSL), which results in a lowering of plasma non-esterified fatty acids (NEFA) which in turn alters hepatic fat metabolism to reduce the output of LDL and VLDL (low and very low density lipoprotein). Reduced VLDL levels are thought to lower cholesterol ester transfer protein (CETP) activity to result in increased HDL (high density lipoprotein) levels which may be the cause of the observed cardiovascular benefits. Thus, nicotinic acid produces a very desirable alteration in lipoprotein profiles; reducing levels of VLDL and LDL whilst increasing HDL. Nicotinic acid has also been demonstrated to have disease modifying benefits, reducing the progression and increasing the regression of atherosclerotic lesions and reducing the number of cardiovascular events in several trials.

The observed inhibition of HSL by nicotinic acid treatment is mediated by a decrease in cellular cyclic adenosine monophosphate (cAMP) caused by the G-protein-mediated inhibition of adenylyl cyclase. Recently, the G-protein coupled receptors HM74 and HM74A have been identified as receptors for nicotinic acid (PCT patent application WO02/84298; Wise et. al. J Biol. Chem., 2003, 278 (11), 9869-9874). The DNA sequence of human HM74A may be found in Genbank; accession number AY148884. Two further papers support this discovery, (Tunaru et. al. Nature Medicine, 2003, 9(3), 352-255 and Soga et. al. Biochem Biophys Res Commun., 2003, 303 (1) 364-369), however the nomenclature differs slightly. In the Tunaru paper what they term human HM74 is in fact HM74A and in the Soga paper HM74b is identical to HM74A. Cells transfected to express HM74A and/or HM74 gain the ability to elicit Gᵢ G-protein mediated responses following exposure to nicotinic acid. In mice lacking the homologue of HM74A (m-PUMA-G) nicotinic acid fails to reduce plasma NEFA levels.

We now present a group of xanthine derivatives which are selective agonists of the nicotinic acid receptor HM74A and are thus of benefit in the treatment, prophylaxis and suppression of diseases where under-activation of this receptor either contributes to the disease or where activation of the receptor will be beneficial.

### SUMMARY OF THE INVENTION

The present invention provides therapeutically active xanthine derivatives and the use of these derivatives in therapy, particularly in the treatment of diseases where under-activation of the HM74A receptor contributes to the disease or where activation of the receptor will be beneficial, in particular diseases of lipid metabolism including dyslipidaemia or hypertripoproteinaemia such as diabetic dyslipidaemia and mixed dyslipidaemia, heart failure, hypercholesteraemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa and obesity. The compounds may also be of use in the treatment of inflammatory diseases or conditions, as set out further below.

Intermediates, formulations, methods and processes described herein form further aspects of the invention.

### DETAILED DESCRIPTION OF THE INVENTION

According to one aspect of this invention, we provide at least one chemical entity selected from compounds of formula (I)

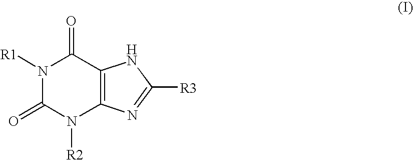

and pharmaceutically acceptable derivatives thereof, wherein
R¹ represents a group selected from: hydrogen, C₁₋₁₀ alkyl, C₂₋₁₀ alkenyl, C₂₋₁₀ alkynyl, and -(alk)ₘ-X-(alk)ₙ-Y,
Wherein X represents A, A1, A2 or a direct link;
A represents a group selected from:
cycloalkylene, cycloalkenylene, aryl, heteroaryl, heterocyclyl, —CH₂—OC(O)—;
A1 represents a group selected from:
—CH₂—O—(CH₂)_{q}aryl-O—, —CH₂—O—(CH₂)_{w}N(R⁵)C(O)O—, —CH₂—N(R⁵)C(O)O—, —CH₂—N(R⁵)C(O)—, —CH₂—(O)ₚ—(CH₂)_{q}C(O)NR⁶—CH₂—N(R⁵)C(O)N(R⁵)—, —CH₂—C(O)N((CH₂)_{w}OH)—, —CH₂—NR⁵—S(O)ᵣ, CH₂—S(O)₂NR⁵—, —CH₂—C(O)O—, —O—, —NR⁵—, —S—;
A2 represents:

### —CH(OH)—;

When X is A, A1 or A2, Y represents a group selected from:
heteroaryl, heterocyclyl, aryl, cycloalkyl, cycloalkenyl, —O(CH₂)₂-aryl, —C(O)—O-aryl, —CH(aryl)₂, —CH(heteroaryl)₂, —C₁₋₄ haloalkyl, —C(O)R^{4,}, —NR⁵R⁷, —C(O)NR⁵R⁷, —NR⁵C(O)R⁷, —NR⁵C(O)OR⁷, —C(O)(CH₂)_{q}OR⁴, halogen, cyano, —N(R⁵)C(O)OR⁷, —OC(O)NR⁵R⁶, —NR⁵C(D)R⁸, —OR⁵, —OC(O)R⁴;
When X is A1 and Y is selected from:
—O(CH₂)ₙ-aryl, —O-heteroaryl, —OR⁵, —OC(O)R⁵, —NH-aryl, —OC(O)NR⁵R⁶,
n is an integer selected from 2, 3, 4 and 5;
When X is A1 and Y is —CF₃, or when X is A2, n is an integer selected from 1, 2, 3, 4 and 5;
When X is a direct link, Y represents a group selected from:
—C(O)(CH₂)_{q}OR⁵, —C(O)-aryl, —C(O)-heteroaryl, —C(O)-heterocyclyl, -hetero aryl, -heterocyclyl, -aryl, -cycloalkyl, -cycloalkenyl, —C₁₋₆ haloalkyl, -halo, -cyano, 3 or 4 ring fused system, —CH(aryl)₂, —CH(heteroaryl)₂, —OR⁵, —NR⁵R⁷, —NCOOR⁸, —(O)ₚC(O)NR⁵R⁶, —NR⁵C(O)R⁸, —OR⁵, —(O)ₚC(O)R⁴;
When Y incorporates a ring, that ring may be optionally substituted by one or more of:
C₁₋₆alkyl, C₂₋₆ alkenyl, C₂₋₆ alkynyl, halogen, —NH₂, (CH₂)_{q}—NR⁵R⁷, —(CH₂)_{q}—(O)ₚ—(CH₂)_{q}—N(R⁵)C(O)OR⁸, (CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q} C(O)N((CH₂)ₘOH)R⁵, —(CH₂)_{q} N(R⁵)—S(O)₂R⁸, —CH₂—S(O)₂N(R⁵)R⁶, —C₁₋₆ haloalkyl, —OCF₃, —OCH(F)₂, —OCH₂F, —COOR⁵, —OR⁵, —(R⁵)ₚCN, —S(O₂)R⁹, —(CH₂)ₙheteroaryl, —(CH₂)ₙheterocycyl, —(CH₂)ₙcycloalkyl, —(CH₂)ₙcycloalkenyl, —(CH₂)ₙaryl;
R² is selected from: hydrogen; or Cl₁₋₀ alkyl, C₂₋₁₀ alkenyl, C₂₋₁₀ alkynyl, cycloalkyl, cycloalkenyl, heterocyclyl, aryl, and heteroaryl, each of which may be optionally substituted by one or more of: C₁₋₁₀ alkyl, C₂₋₁₀ alkenyl, C₂₋₁₀ alkynyl, cycloalkyl, heterocyclyl, aryl, heteroaryl, C₁₋₆ haloalkyl, halogen, —CN, —OR⁴, —(CH₂)ₙCOR⁴, —C(O)OR⁴, —OCOR⁴, —(CH₂)ᵣNR⁵R⁶, —(NH)ₚCONR⁵R⁶, —OCONR⁵R⁷, and —NHC(O)OR⁷;
R³ is selected from: halogenated C₁— alkyl;
R⁴ is selected from: hydrogen, C₁₋₆ alkyl, C₂₋₆ alkenyl, C₂— alkynyl, —(CH₂), cycloalkyl, —(CH₂), cycloalkenyl, —(CH₂)ᵣ heterocyclyl, —(CH₂), aryl, and —(CH₂), heteroaryl;
R⁵ and R⁶ are selected from: hydrogen and C₁₋₄ alkyl;
R⁷ is selected from: hydrogen, C₁₋₆ alkyl, C₂₋₆ alkenyl, C₂₋₆ alkynyl, —(CH₂)ₜ cycloalkyl, —(CH₂), cycloalkenyl, —(CH₂)ₜ heterocyclyl, —(CH₂)ₜ aryl, and —(CH₂)ₜ heteroaryl;
R⁸ is selected from C₁₋₄ alkyl;
R⁹ is selected from Cue alkyl, C₂₋₆ alkenyl, C₂₋₆ alkynyl, —(CH₂)ₙ cycloalkyl, —(CH₂)ₙcycloalkenyl, —(CH₂)ₙheterocyclyl, —(CH₂)ₙaryl, and —(CH₂)ₙheteroaryl, CN;
m represents an integer selected from: 0, 1, 2, 3, 4 and 5;
n represents an integer selected from: 0, 1, 2, 3, 4 and 5;
p represents an integer selected from: 0 and 1;
q represents an integer selected from: 0, 1 and 2;
t represents an integer selected from: 1 and 2;
w represents an integer selected from: 2, 3 and 4.
with the proviso that:
i) when R¹ represents hydrogen or C₁₋₃ alkyl, R² is different to R¹;
ii) when R¹ represents ethyl and R³ represents CF₃, R² is other than CH₂—CH═CH₂; and
iii) when R¹ represents Methyl and R³ represents CF₃, R² is other than i-butyl.

In one embodiment of this invention, we provide at least one chemical entity selected from compounds of formula (I)

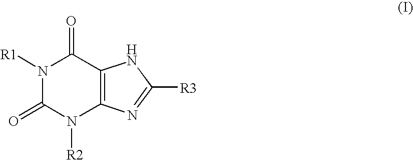

and pharmaceutically acceptable derivatives thereof, wherein
R¹ represents hydrogen or a group selected from: C₁₋₁₀ alkyl, C₂₋₁₀ alkenyl, C₂₋₁₀ alkynyl, —(CH₂)ₙcycloalkyl, —(CH₂)ₙheterocyclyl, —(CH₂)ₙaryl and —(CH₂)ₙheteroaryl, each of which may be optionally substituted by one or more of: —(CH₂)ₙcycloalkyl, —(CH₂)ₙheterocyclyl, —(CH₂)ₙaryl, —(CH₂)ₙheteroaryl, C₁₋₆ haloalkyl, halo, cyano, —OR⁴, —(CH₂)ₙCOR⁴, —CO₂R⁴, —OCOR⁴, —(CH₂)ₙNR⁵R⁷, —(NH)ₘCONR⁵R⁷, —OCONR⁶R⁷, and —NHCO₂R⁷;
R² is selected from: C₁₋₁₀ alkyl, C₂₋₁₀ alkenyl, C₂₋₁₀ alkynyl, —(CH₂)ₙcycloalkyl, —(CH₂)ₙheterocyclyl, —(CH₂)ₙaryl and —(CH₂)ₙheteroaryl, each of which may be optionally substituted by one or more of: cycloalkyl, heterocyclyl, aryl, heteroaryl, C₁₋₆ haloalkyl, halo, cyano, —OR⁴, —(CH₂)ₙCOR⁴, —CO₂R⁴, —OCOR⁴, —(CH₂)ₙNR⁵R⁶, (NH)ₘCONR⁵R⁶, —OCONR⁵R⁷, and —NHCO₂R⁷;
R³ is selected from: halogenated C₁₋₆ alkyl;
R⁴ is selected from: hydrogen, C₁₋₆ alkyl C₂₋₆ alkenyl, C₂₋₆ alkynyl, —(CH₂)ₙcycloalkyl, —(CH₂)ₙheterocyclyl, —(CH₂)ₙaryl, and —(CH₂)ₙheteroaryl;
R⁵ and R⁶ are independently selected from: hydrogen and C₁₋₄ alkyl;
R⁷ is selected from: hydrogen, C₁₋₆ alkyl C₂₋₆ alkenyl, C₂₋₆ alkynyl, —(CH₂)ₚcycloalkyl, —(CH₂)ₚheterocyclyl, —(CH₂)ₚaryl, and —(CH₂)ₚheteroaryl;
n represents an integer selected from: 0, 1, 2, 3 and 4;
m represents an integer selected from: 0 and 1;
p represents an integer selected from: 1 and 2.
with the proviso that:
i) when R¹ represents H or C₁₋₃ alkyl, R² is different to R¹;
ii) when R¹ represents ethyl and R³ represents CF₃, R² is other than CH₂—CH═CH₂; and
iii) when R¹ represents Methyl and R³ represents CF₃, R² is other than i-butyl.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

As used herein, the terms ‘halogen’ or “halo” refer to fluorine, chlorine, bromine and iodine.

As used herein, the term “alkyl” (when used as a group or as part of a group) refers to a straight or branched hydrocarbon chain unless specified otherwise, containing the specified number of carbon atoms. For example, C₃-C₁₀alkyl means a straight or branched hydrocarbon chain containing at least 3 and at most 10 carbon atoms. Examples of alkyl as used herein include, but are not limited to methyl (Me), ethyl (Et), n-propyl and i-propyl.

As used herein, the term “alkenyl” refers to a straight or branched hydrocarbon chain containing the specified number of carbon atoms which contains one or more double bonds. Suitable examples include but are not limited to ethenyl, 2-propenyl, 3-butenyl, 2-butenyl, 2-pentenyl, 3-pentenyl, 3-methyl-2-butenyl, 3-hexenyl and the like.

As used herein, the term “alkynyl” refers to a straight or branched hydrocarbon chain containing the specified number of carbon atoms which contains one or more triple bonds. Suitable examples include but are not limited to acetylenyl, propynyl, 1-butynyl, 1-pentynyl, 3-methyl-1-butynyl and the like.

The term ‘C₁₋₆ haloalkyl’ as used herein refers to a C₁₋₆ alkyl group as defined herein wherein at least one hydrogen atom is replaced with halogen. Examples of such groups include fluoroethyl, trifluoromethyl or trifluoroethyl and the like.

As used herein, the term “cycloalkyl” refers to a hydrocarbon ring, containing between 3 and 6 carbon atoms, comprising no heteroatoms or conjugated double bonds. Examples of cycloalkyl as used herein include, but are not limited to cyclopropyl and cyclohexyl.

The term ‘cycloalkylene’ as used herein refers to a saturated monocyclic hydrocarbon ring of 3 to 8 carbon linker groups. Examples of such groups include cyclopropylene, cyclobutylene, cyclopentylene, cyclohexylene, cycloheptylene or cyclooctylene and the like.

The term ‘cycloalkenyl’ as used herein refers to an unsaturated non-aromatic monocyclic hydrocarbon ring of 3 to 8 carbon atoms containing one or more carbon-carbon double bonds. Examples of such groups include cyclopropenyl, cyclobutenyl, cyolopentenyl, cyclohexenyl, cycloheptenyl or cyclooctenyl and the like.

The term ‘cycloalkenylene’ as used herein refers to an unsaturated non-aromatic monocyclic hydrocarbon ring of 3 to 8 carbon linker groups containing one or more carbon-carbon double bonds. Examples of such groups include cyclopropenylene, cyclobutenylene, cyclopentenylene, cyclohexenylene, cycloheptenylene or cyclooctenylene and the like.

As used herein, the term “aryl” refers to a 5 or 6 membered, monocyclic aromatic group, or a fused 8-10 membered bicyclic aromatic group with at least one ring having a conjugated pi-electron system, containing up to two conjugated or fused ring systems. “Aryl” includes carbocyclic aryl and biaryl groups. Suitable examples include but are not limited to phenyl, naphthyl and the like.

As used herein, the term “heteroaryl” refers to a 5 or 6 membered, monocyclic aromatic group or a fused 8-10 membered bicyclic, aromatic group containing 1 to 3 heteroatoms independently selected from oxygen, nitrogen and sulphur with at least one ring having a conjugated pi-electron system, containing up to two conjugated or fused ring systems. Suitable examples of such monocyclic aromatic rings include but are not limited to thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include but are not limited to benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like.

As used herein, the term “heterocyclyl” refers to a 5 or 6 membered, saturated cyclic hydrocarbon group, containing 1 to 3 heteroatoms independently selected from oxygen, nitrogen and sulphur. Suitable examples include but are not limited to pyrrolidinyl, morpholinyl, imidazolidinyl and piperazinyl.

The term ‘3 or 4 ring fused system’ as used herein refers to a fused 12-18 membered tricyclic or tetracyclic ring which contains 1 to 4 heteroatoms of N and wherein at least one ring is aromatic. There may be one or more optional oxo substituents on the ring carbon atoms. Examples of such fused aromatic rings include carbazolyl, acenaphthyl, naphthotriazolyl and the like.

As used herein, where a group is referred to as being “substituted” by another group or having “one or more substituents” unless a particular position for such a substitution is specified it is to be understood that a substitution may be present at any position in the group.

As used herein, the term “pharmaceutically acceptable derivative” refers to any pharmaceutically acceptable derivative of a compound of the present invention, for example salts, solvates or esters which, upon administration to a mammal, such as a human, are capable of providing (directly or indirectly) a compound of formula (I) or an active metabolite or residue thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

It will be appreciated by those skilled in the art that the compounds of the invention may be modified to provide pharmaceutically acceptable derivatives thereof at any of the functional groups in the compounds, and that the compounds of the invention may be so modified at more than one position.

In one embodiment, “pharmaceutically acceptable derivative” refers to salts or solvates.

As used herein, the term “pharmaceutically acceptable” used in relation to an ingredient (active ingredient or excipient) which may be included in a pharmaceutical formulation for administration to a patient, refers to that ingredient being acceptable in the sense of being compatible with any other ingredients present in the pharmaceutical formulation and not being deleterious to the recipient thereof.

As used herein, the term “solvate” refers to a complex of variable stoichiometry formed by a solute (in this invention, a compound of formula (I), a salt thereof or a pharmaceutically acceptable derivative thereof) and a solvent. Such solvents for the purposes of the present invention may not interfere with the biological activity of the solute. The solvent used may be a pharmaceutically acceptable solvent. Examples of suitable pharmaceutically acceptable solvents include water, ethanol and acetic acid. An example of a solvent that may be used is water, in which case the solvate may be referred to as a hydrate of the solute in question.

It will be appreciated that, for pharmaceutical use, the “salt or solvate” referred to above will be a pharmaceutically acceptable salt or solvate. However, other salts or solvates may find use, for example, in the preparation of a compound of formula (I) or in the preparation of a pharmaceutically acceptable salt or solvate thereof.

Pharmaceutically acceptable salts include those described by Berge, Bighley and Monkhouse, J. Pharm. Sci., 1977, 66, 1-19. Suitable pharmaceutically acceptable salts include alkali metal salts formed from the addition of alkali metal bases such as alkali metal hydroxides. Examples of suitable alkali metal salts are sodium salt or potassium salt. Other suitable pharmaceutically acceptable salts include alkaline earth metal salts such as calcium salt or magnesium salt, ammonium salts; or salts with organic bases such as ethanolamine, triethanolamine, ethylene diamine, triethylmine, choline and meglumine; or salts with amino acids such as arginine, lysine and histidine.

The compounds are of use in the treatment of diseases where under-activation of the HM74A receptor contributes to the disease or where activation of the receptor will be beneficial, in particular diseases of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. As such the compounds of the present invention may find use as agonists or partial agonists of HM74A.

Compounds of the invention are of potential therapeutic benefit in the treatment and amelioration of the symptoms of many diseases of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity. As such, the compounds may also find favour as therapeutics for coronary artery disease, thrombosis, angina, chronic renal failure, peripheral vascular disease and stroke.

Furthermore, it is also believed that the HM74 and HM74A receptors are involved in inflammation. Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation with regards to disease typically is referred to as chronic inflammation and can last up to a lifetime. Such chronic inflammation may manifest itself through disease symptoms. The aim of anti-inflammatory therapy is therefore to reduce this chronic inflammation and allow for the physiological process of healing and tissue repair to progress.

Examples of inflammatory diseases or conditions for which the compounds of the present invention may demonstrate utility include those of the joint, particularly arthritis (e.g. rheumatoid arthritis, osteoarthritis, prosthetic joint failure), or the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes melitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

It will be appreciated that references herein to treatment extend to prophylaxis, prevention of recurrence and suppression of symptoms as well as the treatment of established conditions.

Nicotinic acid has a significant side effect profile, possibly because it is dosed at high level (gram quantities daily). The most common side effect is an intense cutaneous flushing. In certain embodiments of the present invention the compounds may exhibit reduced side effects compared to nicotinic acid. HM74A has been identified as a high affinity receptor for nicotinic acid whilst HM74 is a lower affinity receptor. The compounds of the present invention may find use as selective HM74A agonists or partial agonists; in which case they will show greater affinity for HM74A than for HM74.

The potential for compounds of formula (I) to activate HM74A may be demonstrated, for example, using the following in vitro whole cell assays:

### In-vitro Testing

For transient transfections, HEK293T cells (HEK293 cells stably expressing the SV40 large T-antigen) were maintained in DMEM containing 10% foetal calf serum and 2 mM glutamine. Cells were seeded in 90 mm culture dishes and grown to 60-80% confluence (18-24 h) prior to transfection. Human HM74A (GenBank™ accession number AY148884) was subcloned in to a mammalian expression vector (pcDNA3; Invitrogen) and transfected using Lipofectamine reagent. For transfection, 91 g of DNA was mixed with 30 μl Lipofectamine in 0.6 ml of Opti-MEM (Life Technologies Inc.) and was incubated at room temperature for 30 min prior to the addition of 1.6 ml of Opti-MEM. Cells were exposed to the Lipofectamine/DNA mixture for 5 h and 6 ml of 20% (v/v) foetal calf serum in DMEM was then added. Cells were harvested 48 h after transfection. Pertussis toxin treatment was carried out by supplementation into media at 50 ngml⁻¹ for 16 h. All transient transfection studies involved co-transfection of receptor together with the G_{i/o} G protein, Gₒ₁α.

For generation of stable cell lines the above method was used to transfect CHO-K1 cells seeded in six well dishes grown to 30% confluence. These cells were maintained in DMEM F-12 HAM media containing 10% foetal calf serum and 2 mM glutamine. 48 h post-transfection the media was supplemented with 400 μg/ml Geneticin (G418, Gibco) for selection of antibiotic resistant cells. Clonal CHO-K1 cell lines stably expressing HM74A were confirmed by [³⁵S]-GTPγS binding measurements, following the addition of nicotinic acid.

P2 membrane preparation—Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a Ultra Turrax followed by passage (5 times) through a 25-gauge needle. Cell lysates were centrifuged at 1,000 g for 10 min in a microcentrifuge to pellet the nuclei and unbroken cells and P2 particulate fractions were recovered by microcentrifugation at 16,000 g for 30 min. P2 particulate fractions were resuspended in buffer A and stored at 80° C. until required.

[³⁵S]-GTPγS binding—assays were performed at room temperature in 384-well format based on methods described previously, (Wieland, T. and Jakobs, K. H. (1994) Methods Enzymol. 237, 3-13). Briefly, the dilution of standard or test compounds were prepared and added to a 384-well plate in a volume of 10 μl. Membranes (HM74A or HM74) were diluted in assay buffer (20 mM HEPES, 100 mM NaCl, 10 mM MgCl₂, pH7.4) supplemented with saponin (60 μg/ml), Leadseeker WGA beads (Amersham; 250 μg/well) and 10 μM GDP, so that the 20 μl volume added to each well contains 5 μg of membranes. [³⁵S]-GTPγS (1170 Ci/mmol, Amersham) was diluted (1:1500) in assay buffer and 20 μl added to each well. Following the addition of the radioligand, the plates were sealed, pulse spun and incubated for 4 hours at room temperature. At the end of the incubation period the plates were read on a Leadseeker machine (VIEWLUX PLUS; Perkin-Elmer) to determine the levels of specific binding.

These assays were refined by reducing the final assay volume to 10 μl. For this 10 μl assay a revised protocol was used. This involved the use of only 100 nl of standard or test compound per well of a 384-well plate and 1.5 μg membrane and 100 μg Leadseeker WGA beads. For the low volume protocol, membrane, beads and [³⁵S]-GTPγS were mixed together and then 10 μl of this mix were dispensed to each well. Incubation and plate read were identical for the 10 μl and 50 μl assays.

All exemplified compounds were tested in one or both of the [³⁵S]-GTPγS binding assays described above (i.e. the 10 and 50 μl assays).

Data was analysed by curve fitting as carried out using a Four Parameter Logistical equation using the XC50 software package (max 2 points deleted from any one curve). Specific binding is expressed as pEC₅₀ and as % efficacy compared to the maximal response of nicotinic acid binding.

### In-vivo Testing

HM74A agonists can be tested in male Spague-Dawley rats (200-250 g) which have been fasted for at least 12 hours prior to the study. The compounds are dosed intravenously at either 1 or 3 mg/kg (5 ml/kg) or by oral gavage at doses ranging from 1-30 mg/kg (10 ml/kg). Blood samples (0.3 ml tail vein bleed) can be taken pre-dose and at three times post-dose (times ranging from 15 minutes to 6 hours post-dose). Each blood sample is transferred to a heparin tube (Becton Dickinson Microtainer, PST LH) and centrifuged (10,000 g for 5 minutes) to produce a plasma sample. The plasma samples are assayed for levels of non-esterified fatty acids (NEFA) using a commercially available kit (Randox). Inhibition of plasma NEFA levels, relative to pre-dose levels, are used as a surrogate for HM74A agonist activity.

In order to determine whether HM74A compounds exhibit the flushing response associated with nicotinic acid they can be dosed to conscious guinea-pigs. Male Dunkin Hartley guinea pigs (300-600 g; n=10-20 per group) are fasted for at least 12 hours, but not in excess of 24 hours prior to experimention. Pre-study blood samples (0.5 ml) are taken from each animal by cardiac puncture under recovery anaesthesia (Isoflurane 3.5% with additional O2 (1 L/min)). Ear temperature measurements are taken by placing the left ear of each animal over an infra-red temperature probe. Measurements are taken at one minute intervals from 5 minutes pre-dose to 30 minutes post-dose. Temperature measurements are then taken at 15 minute intervals up to 2 hours post-dose. Animals receive test compounds by oral gavage (5 ml/kg). Blood samples (0.5 ml) are taken by cardiac puncture under terminal anaesthesia. Blood samples are taken from individual animals to provide data at 0.5, 1, 2, 3, and 4 hours post-dose. All blood samples are placed on a blood roller for 5 minutes then stored on ice until the end of the study. Following centrifugation (12000 g for 5 min) the plasma is transferred into fresh tubes and stored at −20° C. until assayed for NEFA concentrations.

Compounds according to Formula (I) have been synthesised (see synthetic examples below) and tested in the [³⁵S]-GTPγS binding assays described above. All of the exemplified compounds have a pEC50 of 4.3 or greater and an efficacy of 30% or greater (in relation to nicotinic acid).

Compounds of Formula (I) may find use in human or veterinary medicine, in particular as activators of HM74A, in the management of dyslipidaemia and hyperlipoproteinaemia.

According to another aspect of the invention, there is provided the use of at least one chemical entity selected from compounds of formula (II)

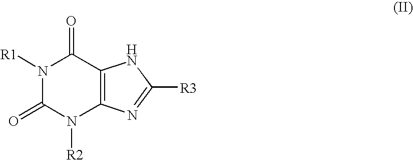

and pharmaceutically acceptable derivatives thereof, wherein
R¹ represents a group selected from: hydrogen, C₁₋₁₀ alkyl, C₂₋₁₀ alkenyl, C₂₋₁₀ alkynyl, and -(alk)ₘ-X-(alk)ₙ-Y,
Wherein X represents A, A1, A2 or a direct link;
A represents a group selected from:
cycloalkylene, cycloalkenylene, aryl, heteroaryl, heterocyclyl, —CH₂—OC(O)—;
A1 represents a group selected from:
—CH₂—O—(CH₂)_{q}aryl-O—, —CH₂—O—(CH₂)_{w}N(R⁵)C(O)O—, —CH₂—N(R⁵)C(O)O—, —CH₂—N(R⁵)C(O)—, —CH₂—(O)ₚ—(CH₂)_{q}C(O)NR⁵—, —CH₂—N(R⁵)C(O)N(R⁵)—, —CH₂—C(O)N((CH₂)_{w}OH)—, —CH₂—NR⁵—S(O)₂—, CH₂—S(O)₂NR⁵—, —CH₂—C(O)O—, —O—, —NR⁵—, —S—;
A2 represents:

### —CH(OH)—;

When X is A, A1 or A2, Y represents a group selected from: heteroaryl, heterocyclyl, aryl, cycloalkyl, cycloalkenyl, —O(CH₂)ₙ-aryl, —C(O)O-aryl, —CH(aryl)₂, —CH(heteroaryl)₂, —C₁₋₈ haloalkyl, —C(O)R⁴, —NR⁵R⁷, —C(O)NR⁵R⁷, —NR⁵C(O)R⁷, —NR⁵C(O)OR⁷, —C(O)(CH₂)_{q}OR⁴, halogen, cyano, —N(R⁵)C(O)OR⁷, —OC(O)NR⁵R⁶, —NR⁵C(O)R⁸, —OR⁵, —OC(O)R⁴;
When X is A1 and Y is selected from:
—O(CH₂)ₙ-aryl, —O-heteroaryl, —OR⁵, —OC(O)R⁵, —NH-aryl, —OC(O)NR⁵R⁶,
n is an integer selected from 2, 3, 4 and 5;
When X is A1 and Y is —CF₃, or when X is A2, n is an integer selected from 1, 2, 3, 4 and 5;
When X is a direct link, Y represents a group selected from:
—C(O)(CH₂)_{q}OR⁵, —C(O)-aryl, —C(O)-heteroaryl, —C(O)-heterocyclyl, -heteroaryl, -heterocyclyl, -aryl, -cycloalkyl, -cycloalkenyl, —C₁₋₆ haloalkyl, -halo, -cyano, 3 or 4 ring fused system, —CH(aryl)₂, —CH(heteroaryl)₂, —OR⁵, —NR⁵R⁷, —NCOOR⁸, —(O)ₚC(O)NR⁵R⁶, —NR⁵C(O)R⁸, —OR⁵, —(O)ₚC(O)R⁴;
When Y incorporates a ring, that ring may be optionally substituted by one or more of:
C₁₋₄alkyl, C₂₋₆ alkenyl, C₂₋₆ alkynyl, halogen, —NH₂, (CH₂)_{q}—NR⁵R⁷, —(CH₂)_{q}—(O)ₚ—(CH₂)_{q}—N(R⁵)C(O)OR⁸, (CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —C₁₋₆ haloalkyl, —OCF₃, —OCH(F)₂, —OCH₂F, —COOR⁵, —OR⁵, —(R⁸)ₚCN, —S(O₂)R⁹, —(CH₂)ₙheteroaryl, —(CH₂)ₙheterocycyl, —(CH₂)ₙcycloalkyl, —(CH₂)ₙcycloalkenyl, —(CH₂)ₙaryl;
R² is selected from: hydrogen; or C₁₋₁₀ alkyl, C₂₋₁₀ alkenyl, C₂₋₁₀ alkynyl, cycloalkyl, cycloalkenyl, heterocyclyl, aryl, and heteroaryl, each of which may be optionally substituted by one or more of: C₁₋₁₀ alkyl, C₂₋₁₀ alkenyl, C₂₋₁₀ alkynyl, cycloalkyl, heterocyclyl, aryl, heteroaryl, C₁₋₄ haloalkyl, halogen, —CN, —OR⁴, —(CH₂)ₙCOR⁴, —C(O)OR⁴, —OCOR⁴, —(CH₂)ₙNR⁵R⁶, —(NH)ₚCONR⁵R⁶, —OCONR⁵R⁷, and —NHC(O)OR⁷;
R³ is selected from: halogenated C₁₋₆ alkyl;
R⁴ is selected from: hydrogen, C₁₋₄ alkyl, C₂₋₄ alkenyl, C₂₋₆ alkynyl, —(CH₂), cycloalkyl, —(CH₂), cycloalkenyl, —(CH₂)ₙ heterocyclyl, —(CH₂)ₙaryl, and —(CH₂)ₙheteroaryl;
R⁵ and R⁶ are selected from: hydrogen and C₁₋₄ alkyl;
R⁷ is selected from: hydrogen, C₁₋₆ alkyl, C₂₋₆ alkenyl, C₂₋₆ alkynyl, —(CH₂)ₜ cycloalkyl, —(CH₂)ₙ cycloalkenyl, —(CH₂)ₜ heterocyclyl, —(CH₂)ₜaryl, and —(CH₂)ₜ heteroaryl;
R⁸ is selected from C₁₋₄ alkyl;
R⁹ is selected from C₁₋₆ alkyl, C₂₋₆ alkenyl, C₂₋₄ alkynyl, —(CH₂)ₙ cycloalkyl, —(CH₂)ₙcycloalkenyl, —(CH₂)ₙheterocyclyl, —(CH₂)ₙaryl, and —(CH₂)ₙheteroaryl, CN;
m represents an integer selected from: 0, 1, 2, 3, 4 and 5;
n represents an integer selected from: 0, 1, 2, 3, 4 and 5;
p represents an integer selected from: 0 and 1;
q represents an integer selected from: 0, 1 and 2;
t represents an integer selected from: 1 and 2;
w represents an integer selected from: 2, 3 and 4;
in the manufacture of a medicament for the treatment of disorders of lipid metabolism including dyslipidaemia or hyperlipoproteinaemia. In particular, the use is provided of a compound of Formula (II) in the manufacture of a medicament for the treatment of diabetic dyslipidaemia or mixed dyslipidaemia, heart failure, hypercholesteraemia, type II diabetes mellitus, type I diabetes, insulin resistance, hyperlipidaemia, anorexia nervosa, obesity, coronary artery disease, thrombosis, angina, chronic renal failure, stroke and cardiovascular disease including atherosclerosis, arteriosclerosis, and hypertriglyceridaemia.

In one embodiment of the invention, there is provided a compound of formula (I) or (II) or a pharmaceutically derivative thereof for use in the treatment of disorders of lipid metabolism including dyslipidaemia or hyperlipoproteinaemia. In another embodiment, the use is provided of a compound of Formula (I) or (II) in the manufacture of a medicament for the treatment of diabetic dyslipidaemia or mixed dyslipidaemia, heart failure, hypercholesteraemia, type II diabetes mellitus, type I diabetes, insulin resistance, hyperlipidaemia, anorexia nervosa, obesity, coronary artery disease, thrombosis, angina, chronic renal failure, stroke and cardiovascular disease including atherosclerosis, arteriosclerosis, and hypertriglyceridaemia.

Thus, there is provided as a further aspect of the present invention a compound of formula (I) or (II) or a pharmaceutically acceptable derivative thereof, for use in human or veterinary medicine, particularly in the treatment of disorders of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity. As such, the compounds are also provided for use in the treatment of coronary artery disease, thrombosis, angina, chronic renal failure, peripheral vascular disease and stroke.

There is provided as a further aspect of the present invention a compound of formula (I) or (II) or a pharmaceutically acceptable derivative thereof, for use in the manufacture of a medicament for the treatment of disorders of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity. As such, the compounds are also provided for use in the treatment of coronary artery disease, thrombosis, angina, chronic renal failure, peripheral vascular disease and stroke.

In a further embodiment the present invention provides the use of a compound of formula (I) or (II) or a pharmaceutically acceptable derivative thereof, in the manufacture of a medicament for the treatment of inflammatory diseases or conditions of the joint, particularly arthritis (e.g. rheumatoid arthritis, osteoarthritis, prosthetic joint failure), or of the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes melitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In one embodiment, the compounds of formula (I) or (II) or pharmaceutically acceptable derivatives thereof are useful in the treatment and prevention of inflammation, diabetes and cardiovascular diseases or conditions including atherosclerosis, arteriosclerosis, hypertriglyceridemia, and mixed dyslipidaemia.

In a further or alternative aspect there is provided a method for the treatment of a human or animal subject with a condition where under-activation of the HM74A receptor contributes to the condition or where activation of the receptor will be beneficial, which method comprises administering to said human or animal subject an effective amount of a compound of formula (I) or (II) or a pharmaceutically acceptable derivative thereof.

In one embodiment the present invention provides a method for the treatment of disorders of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity, which method comprises administering to said human or animal subject an effective amount of a compound of formula (I) or (II) or a pharmaceutically acceptable derivative thereof. As such, these compounds may also find favour in methods for the treatment of coronary artery disease, thrombosis, angina, chronic renal failure, peripheral vascular disease and stroke, which methods comprise administering to said human or animal subject an effective amount of a compound of formula (I) or (II) or a pharmaceutically acceptable derivative thereof.

In one embodiment of formula (I) or (II), R¹ is selected from hydrogen C₁₋₁₀ alkyl, and -(alk)ₘ-X-(alk)ₙ-Y.

In one embodiment of formula (I) or (II), X represents A or A1. In a further embodiment of formula (I) or (II), A is selected from heteroaryl, heterocyclyl, and A1 is selected from CH₂—O—(CH₂)_{w}N(R⁵)C(O)O—, for example CH₂—O—(CH₂)₂NHC(O)O—, CH₂—N(R⁵)C(O)O— for example CH₂—NHC(O)O, CH₂—N(R⁵)C(O)— for example, CH₂—NHC(O)—, CH₂—(O)ₚ—(CH₂)_{q}C(O)NR⁵— for example CH₂C(O)NCH₃—, CH₂—N(R⁵)C(O)N(R⁵)— for example CH₂NHC(O)NCH₃—, CH₂—C(O)N((CH₂)_{w}OH)— for example CH₂—C(O)N((CH₂)₂OH)—, CH₂—NR⁵—S(O)₂— for example CH₂—NH—S(O)₂—, CH₂—S(O)₂NR⁵— for example CH₂—S(O)₂NCH₃—, and CH₂—C(O)O—. In another embodiment of formula (I) or (II), X represents A and A represents a heteroaryl. In another embodiment of formula (I) or (II), A represents a heteroaryl comprising a nitrogen heteroatom, for example, triazolyl, furazanyl, oxadiazolyl, tetrazolyl, imidazolyl or pyrazolyl.

In a further embodiment of formula (I) or (II), X represents A, for example heteroaryl or heterocyclyl, or a direct link.

In one embodiment of formula (I) or (II), Y represents an optionally substituted group selected from: aryl, for example phenyl or napthyl, heteroaryl, for example pyridinyl, thiazolyl, thienyl, benzofuranyl or indolyl, and O-aryl, for example O-phenyl.

In a further embodiment of formula (I) or (II), Y is substituted by one or more groups selected from OR⁵ for example OH or OCH₃, halogen, for example F or Cl, aryl, for example phenyl, C₁₋₆ haloalkyl for example CF₃ or CH₂CF₃, OCF₃, (R⁸)ₚCN for example CN, (CH₂)_{q}—N(R⁵)—S(O)₂R⁸ for example NHS(O)₂CH₃ and S(O)₂R⁹ for example S(O)₂CH₃.

In one embodiment of formula (I) or (II), when X represents A or A1, and A represents cycloalkyl, cycloalkenyl, aryl, heteroaryl or heterocyclyl, and A1 represents —CH₂—O—(CH₂)_{w}N(R⁵)C(O)O—, —CH₂—N(R⁵)C(O)O—, —CH₂—N(R⁵)C(O)—, —CH₂—(O)ₚ—(CH₂)_{q}C(O)NR⁵—, —CH₂—N(R⁵)C(O)N(R⁵)—, or —CH₂—C(O)N((CH₂)_{w}OH)—, and Y represents a ring, for example when X represents oxadizolyl, tetrazolyl or pyrazolyl and Y represents phenyl, pyridinyl, or thienyl, m is an integer selected from 3 and 4 and n is an integer selected from 0 and 1, for example m is 4 and n is 0, or m is 3 and n is 1;

In one embodiment of formula (I) or (II), R¹ is selected from: hydrogen; and C₁₋₁₀ alkyl which may be optionally substituted by one or more of: cycloalkyl, heterocyclyl, aryl, heteroaryl, C₁₋₆ haloalkyl, halo, cyano, —OR⁴, —(CH₂)ₙCOR⁶, —CO₂R⁴, —OCOR⁴, —(CH₂)ₙNR⁵R⁷, and —(NH)ₘCONR⁵R⁷. In another embodiment of formula (I) or (II), R¹ is selected from: hydrogen; and C₁₋₁₀ alkyl which may be optionally substituted by one or more of: halo, cyano, —OR⁴, —(CH₂)ₙCOR⁴, —CO₂R⁴, —OCOR⁴, —(CH₂)ₙNR⁵R⁶, and —(NH)ₘCONR⁵R⁶.

In a further embodiment of formula (I) or (II), R¹ is selected from: hydrogen and C₁₋₆ alkyl optionally substituted with —OH, for example methyl, butyl or CH₂CH₂OH.

In one embodiment of formula (I) or (II), R¹ is selected from: hydrogen and C₁₋₆ alkyl, for example methyl or butyl.

In one embodiment of formula (I) or (II), R² is selected from: C₄₋₁₀ alkyl and C₂₋₁₀ alkenyl, each of which may be optionally substituted by one or more of cycloalkyl, heterocyclyl, aryl, heteroaryl, C₁₋₆ haloalkyl, halo, cyano, —OR⁴, —(CH₂)ₙCOR⁵, —CO₂R⁴, —OCOR⁴, —(CH₂)ₙNR⁵R⁶, and —(NH)ₘCONR⁵R⁶. In another embodiment of formula (I) or (II), R² may represent C₄₋₁₀ alkyl which may be optionally substituted by one or more of cycloalkyl, heterocyclyl, aryl, heteroaryl, C₁₋₆ haloalkyl, halo, cyano, —OR⁴, —(CH₂)ₙCOR⁴, —CO₂R⁴, —OCOR⁴, —(CH₂)ₙNR⁵R⁶, and

—(NH)ₘCONR⁵R⁶. In a further embodiment R² is selected from C₄₋₆ alkyl, for example butyl or pentyl.

In one embodiment of formula (I) or (II), R² represents C₁₋₁₀ alkyl which may be optionally substituted by one or more of: cycloalkyl, heterocyclyl, aryl, heteroaryl, C₁₋₆ haloalkyl, halogen, —CN, —OR⁴, —(CH₂)ₙCOR⁵, —C(O)OR⁴, —OCOR⁴, —(CH₂)ₙNR⁵R⁶, and —(NH)ₚCONR⁵R⁶.

In a further embodiment of formula (I) or (II), R³ is selected from C₁₋₆alkyl, for example butyl or pentyl.

In one embodiment of formula (I) or (II), R³ is selected from: fluorinated C₁₋₆ alkyl. In another embodiment of formula (I) or (II), R³ is selected from: fluorinated C₁₋₄ alkyl, for example CF₃, CHF₂ or CF₂CF₃.

In one embodiment of formula (I) or (II), R⁴ is selected from: hydrogen, C₁₋₆ alkyl, C₂₋₆ alkenyl, C₂₋₆ alkynyl, —(CH₂)ₙcycloalkyl, —(CH₂)ₙheterocyclyl, —(CH₂)ₙaryl, and —(CH₂)ₙheteroaryl. In another embodiment of formula (I) or (II) R⁴ is selected from: hydrogen and C₁₋₄ alkyl.

In one embodiment of formula (I) or (II), R¹ is selected from: hydrogen and C₁₋₆ alkyl optionally substituted with —H, R² is selected from: C₄₋₆ alkyl and R³ is selected from: fluorinated C₁₋₄ alkyl, with the proviso that when R¹ represents Methyl and R³ represents CF₃, R² cannot be i-butyl.

In one embodiment of formula (I) or (II), R⁵ is hydrogen.

In one embodiment of formula (I) or (II), R⁷ is selected from: hydrogen and C₁₋₄ alkyl.

In further embodiment of formula (I) or (II,) it is provided that when X is A1, A1 is —O— and Y is a ring which is substituted by aryl or heteroaryl, then m is an integer selected from 3, 4 and 5.

In one embodiment of formula (I) or (II), R¹ and R² are different.

It is to be understood that the present invention includes any combination of particular embodiments and covers all combinations of particular substituents described hereinabove for compounds of Formula (I) or (II).

Particular compounds of the present invention include:
3-butyl-1-methyl-8-(trifluoromethyl)-3,7-dihydro-1H-purine-2,6-dione,
1,3-dibutyl-8-(trifluoromethyl)-3,7-dihydro-1H-purine-2,6-dione,
3-butyl-1-methyl-8-(pentafluoroethyl)-3,7-dihydro-1H-purine-2,6-dione,
8-(difluoromethyl)-1-methyl-3-pentyl-3,7-dihydro-1H-purine-2,6-dione,
3-pentyl-8-(trifluoromethyl)-3,7-dihydro-1H-purine-2,6-dione,
1-(2-hydroxyethyl)-3-pentyl-8-(trifluoromethyl)-3,7-dihydro-1H-purine-2,6-dione,
3-Butyl-1-{3[3-(phenylmethyl)-1,2,4-oxadiazol-5-yl]propyl}-8-(trifluoromethyl)-3,7-dihydro-1H-purine-2,6-dione,
4-[3-Butyl-2,6-dioxo-8-(trifluoromethyl)-2,3,6,7-tetrahydro-1H-purin-1-yl]butanoic acid,
3-Butyl-1-[4-(3-phenyl-1,2,4-oxadiazol-5-yl)butyl]-8-(trifluoromethyl)-3,7-dihydro-1H-purine-2,6-dione,
5-[3-butyl-2,6-dioxo-8-(trifluoromethyl)-2,3,6,7-tetrahydro-1H-purin-1-yl]pentanoic acid,
3-Butyl-1-{4-[3-(2-fluorophenyl)-1,2,4-oxadiazol-5-yl]butyl}-8-(trifluoromethyl)-3,7-dihydro-1H-purine-2,6-dione,
3-Butyl-1-4-[3(4-fluorophenyl)-1,2,4-oxadiazol-5-yl]butyl)-8-(trifluoromethyl)-3,7-dihydro-1H-purine-2,6-dione,
3-Butyl-1-{4-[3-(2-pyridinyl)-1,2,4-oxadiazol-5-yl]butyl}-8-(trifluoromethyl)-3,7-dihydro-1H-purine-2,6-dione,
3-Butyl-8-(trifluoromethyl)-1-(3-{3-[(2,4,6-trifluorophenyl)methyl]-1,2,4-oxadiazol-5-yl}propyl)-3,7-dihydro-1H-purine-2,6-dione,
1-Methyl-3-pentyl-8-(trifluoromethyl)-3,7-dihydro-1H-purine-2,6-dione.

The amount of a HM74A modulator which is required to achieve the desired biological effect will, of course, depend on a number of factors, for example, the mode of administration and the precise clinical condition of the recipient. In general, the daily dose will be in the range of 0.1 mg-1 g/kg, typically 0.1-100 mg/kg. An intravenous dose may, for example, be in the range of 0.01 mg to 0.1 g/kg, typically 0.01 mg to 10 mg/kg, which may conveniently be administered as an infusion of from 0.1 μg to 1 mg, per minute. Infusion fluids suitable for this purpose may contain, for example, from 0.01 μg to 0.1 mg, per millilitre. Unit doses may contain, for example, from 0.01 μl to 1 g of a HM74A modulator. Thus ampoules for injection may contain, for example, from 0.01 μg to 0.1 g and orally administrable unit dose formulations, such as tablets or capsules, may contain, for example, from 0.1 mg to 1 g. No toxicological effects are indicated/expected when a compound of the invention is administered in the above mentioned dosage range.

A compound of the present invention may be employed as the compound per se in the treatment of a disease where under-activation of the HM74A receptor contributes to the disease or where activation of the receptor will be beneficial, an example of this is where a compound of the present invention is presented with an acceptable carrier in the form of a pharmaceutical formulation. The carrier must, of course, be acceptable in the sense of being compatible with the other ingredients of the formulation and must not be deleterious to the recipient. The carrier may be a solid or a liquid, or both, and may be formulated with the HM74A modulator as a unit-dose formulation, for example, a tablet, which may contain from 0.05% to 95% by weight of the HM74A modulator.

The formulations include those suitable for oral, rectal, topical, buccal (e.g. sub-lingual) and parenteral (e.g. subcutaneous, intramuscular, intradermal or intravenous) administration.

There is also provided according to the invention a process for preparation of such a pharmaceutical composition which comprises mixing the ingredients.

Formulations suitable for oral administration may be presented in discrete units, such as capsules, cachets, lozenges or tablets, each containing a predetermined amount of a HM74A modulator; as a powder or granules; as a solution or a suspension in an aqueous or non-aqueous liquid; or as an oil-in-water or water-in-oil emulsion. In general, the formulations are prepared by uniformly and intimately admixing the active HM74A modulator with a liquid or finely divided solid carrier, or both, and then, if necessary, shaping the product. For example, a tablet may be prepared by compressing or moulding a powder or granules of the HM74A modulator optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Moulded tablets may be made by moulding, in a suitable machine, the powdered compound moistened with an inert liquid diluent.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

Formulations suitable for buccal (sub-lingual) administration include lozenges comprising a HM74A modulator in a flavoured base, usually sucrose and acacia or tragacanth, and pastilles comprising the HM74A modulator in an inert base such as gelatin and glycerin or sucrose and acacia.

Formulations for oral administration may be suitably formulated to give controlled/extended release of the active compound.

Formulations of the present invention suitable for parenteral administration conveniently comprise sterile aqueous preparations of an HM74A modulator, the formulation may be isotonic with the blood of the intended recipient. These preparations could be administered intravenously, although administration may also be effected by means of subcutaneous, intramuscular, or intradermal injection. Such preparations may conveniently be prepared by admixing the HM74A modulator with water and rendering the resulting solution sterile and isotonic with the blood. Injectable compositions according to the invention will generally contain from 0.1 to 5% w/w of the HM74A modulator.

Thus, formulations of the present invention suitable for parenteral administration comprising a compound according to the invention may be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

Formulations suitable for rectal administration may be presented as unit-dose suppositories. These may be prepared by admixing a HM74A modulator with one or more conventional solid carriers, for example, cocoa butter or glycerides and then shaping the resulting mixture.

Formulations suitable for topical application to the skin may take the form of an ointment, cream, lotion, paste, gel, spray, aerosol, or oil. Carriers which may be used include vaseline, lanolin, polyethylene glycols, alcohols, and combinations of two or more thereof. The HM74A modulator is generally present at a concentration of from 0.1 to 15% w/w of the composition, for example, from 0.5 to 2%.

By topical administration as used herein, we include administration by insufflation and inhalation. Examples of various types of preparation for topical administration include ointments, creams, lotions, powders, pessades, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

The pharmaceutical compositions according to the invention may also be used in combination with other therapeutic agents, for example in combination with other classes of dyslipidaemic drugs (e.g. statins, fibrates, bile-acid binding resins or nicotinic acid).

The compounds of the instant invention may be used in combination with one or more other therapeutic agents for example in combination with other classes of dyslipidaemic drugs e.g. 3-hydroxy-3-methylglutaryl-coenzyme A reductase inhibitors (statins) or fibrates or bile acid binding resins or nicotinic acid. The invention thus provides, in a further aspect, the use of such a combination in the treatment of diseases where under-activation of the HM74A receptor contributes to the disease or where activation of the receptor will be beneficial and the use of a compound of formula (I) or (II), or a pharmaceutically acceptable salt, solvate or pharmaceutically acceptable derivative thereof in the manufacture of a medicament for the combination therapy of disorders of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa or obesity.

When the compounds of the present invention are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for such compounds in the art.

When in combination with a second therapeutic agent active against the same disease, the dose of each component may differ from that when the compound is used alone. Appropriate doses will be readily appreciated by those skilled in the art.

The invention thus provides, in a further aspect, a combination comprising a compound of formula (I) or a pharmaceutically acceptable derivative thereof together with another therapeutically active agent.

The combination referred to above may conveniently be presented for use in the form of a pharmaceutical formulation and thus pharmaceutical formulations comprising a combination as defined above together with a pharmaceutically acceptable diluent, excipient or carrier thereof represent a further aspect of the invention. For example, a pharmaceutical formulation comprising a compound or pharmaceutically acceptable derivative thereof and one or more pharmaceutically acceptable diluents, excipients or carriers.

### General Purification and Analytical Methods:

The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES−ve to give (M−H)⁻ molecular ion] modes.

¹H NMR spectra were recorded using a Bruker DPX 400 MHz spectrometer using tetramethylsilane as the external standard.

Biotage™ chromatography refers to purification carried out using equipment sold by Dyax Corporation (either the Flash 40i or Flash 150i) and cartridges pre-packed with KPSil.

Mass directed autoprep refers to methods where the material was purified by high performance liquid chromatography on a HPLCABZ+5 μm column (5 cm×10 mm i.d.) with 0.1% HCO₂H in water and 95% MeCN, 5% water (0.5% HCO₂H) utilising the following gradient elution conditions: 0-1.0 minutes 0.5% B, 1.0-8.0 minutes 5→3D % B, 8.0-8.9 minutes 30% B, 8.9-9.0 minutes 30→95% B, 9.0-9.9 minutes 95% B, 9.9-10 minutes 95→0% B at a flow rate of 8 ml minutes⁻¹ (System 2). The Gilson 202-fraction collector was triggered by a VG Platform Mass Spectrometer on detecting the mass of interest.

Preparative h.p.l.c refers to methods where the material was purified by high performance liquid chromatography on a HPLCABZ+5 μm column (10 cm×21.2 mm i.d.) with 0.1% HCO₂H in water (A) and MeCN (0.5% HCO₂H) (B) utilising the gerienc gradient elution conditions expressed as “x to y” gradient with a gradient system as follows: 0-1.45 minutes x % B, 1.45-20 minutes x→y % B, 20-24 minutes y→95% B, 24-30 minutes 95% B, 32-34 minutes 95→x % B at a flow rate of 8 ml minutes⁻¹. The Gilson 233 fraction collector was triggered by UV (254 nm).

SPE (solid phase extraction) refers to the use of cartridges sold by International Sorbent Technology Ltd.

Strata Phenyl SPE refers to the use of cartridges sold by Phenomenex. The compound was loaded onto a cartridge previously conditioned with MeCN and equilibrated with 5% MeCN in water. The compound was eluted with 0.1% HCO₂H in water and MeCN (0.5% HCO₂H) in a suitable gradient on a Combiflash Optix 10.

The compounds of the present invention and pharmaceutically derivatives thereof may be prepared by the methodology described hereinafter, constituting a further aspect of this invention.

### Process A:

A process according to the invention for preparing a compound of formula (I) or formula (II) in which R¹ is H or alkyl wherein R¹ and R² may be the same or different, comprises:

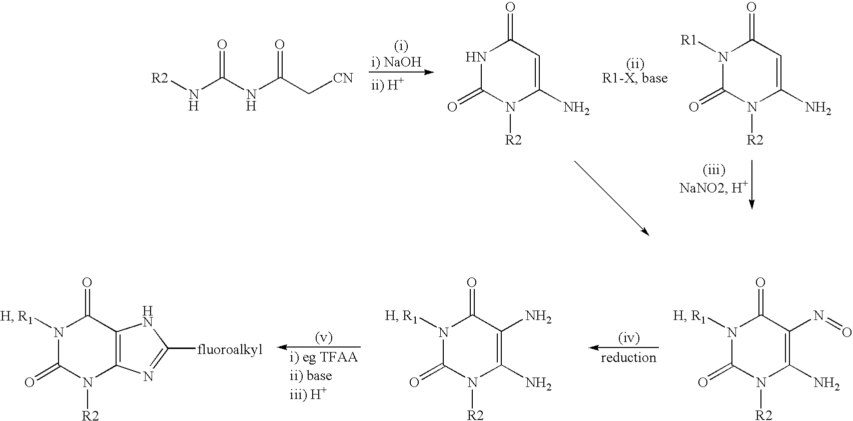

i) Pyrimidinedione formation

### ii) Alkylation

iii) Nitrosation

### iv) Reduction

v) Xanthine formation

### Process B:

A process according to the invention for preparing a compound of formula (I) or formula (II) in which R¹ and R² may be the same or different, comprises:

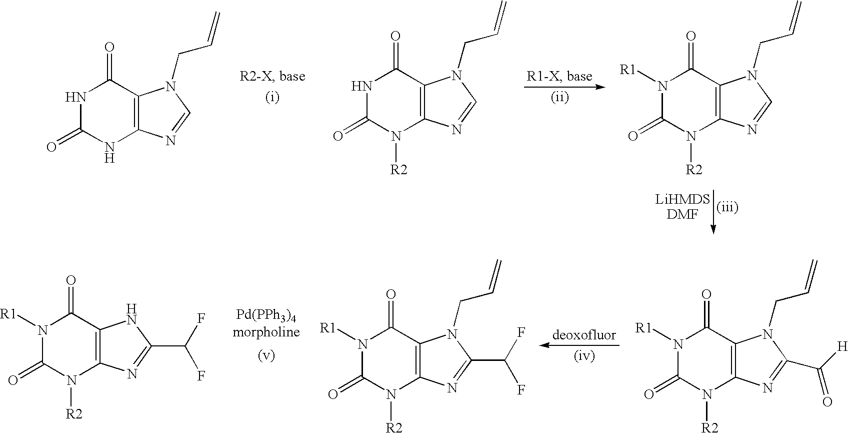

i) Selective alkylation at N3

### ii) Alkylation at N1

iii) Formylation of C8
iv) Fluorination of carbonyl
v) Palladium mediated deprotection

Where desired or necessary, as a final stage in any of the above synthetic processes, a resultant compound of formula (I) or (II) can be converted into a pharmaceutically acceptable salt form or vice versa or converting one salt form into another pharmaceutically acceptable salt form.

### Abbreviations

AcOH Acetic acid
br Broad (NMR)
CDI Carbonyldiimidazole
d Doublet (NMR)
DBAD Di-t-butylazodicarboxylate
DCM Dichloromethane
DIPEA Diisopropylethylamine
DMSO Dimethylsulfoxide
DMF N,N-Dimethylformamide
EtOAc Ethyl acetate
EtOH Ethanol
h Hour(s)
IPA Isopropyl alcohol
m Multiplet (NMR)
MDAP Mass directed autoprep
MeCN Acetonitrile
MeOH Methanol
min Minute(s)
NCS N-Chlorosuccinimide
PFPA pentafluoropropionic anhydride
q Quartet (NMR)
rt Room temperature
RT Retention time
Singlet (NMR)
SPE Solid phase extraction cartridge
t Triplet (NMR)
TFA Trifluoroacetic acid
TFAA Trifluoroacetic acid anhydride
THF Tetrahydrofuran
DMEM Dulbecco's Modified Eagle's Medium
HEPES 4-(2-Hydroxyethyl)piperazine-1-ethanesulphonic acid
LiHMDS Lithium hexamethyldisilylamide

The following non-limiting examples illustrate the present invention:

### SYNTHETIC EXAMPLES

### Example 1

### 3-butyl-1-methyl-8-(trifluoromethyl)-3,7-dihydro-1H-purine-2,6-dione

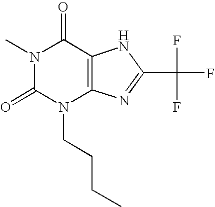

A purple mix of 5,6-diamino-1-butyl-3-methyl-2,4(1H,3H)-pyrimidinedione (European Journal of Medicinal Chemistry (1990), 25(8), 653-8)(290 mg) and TFM (6 ml) was heated at reflux for 1 hour then concentrated in vacuo. The resulting residue was treated with 2N NaOH (aq) (7 ml) and the mixture heated at reflux for 30 minutes. After cooling to ambient temperature, the mixture was acidified to ca. pH4 with 3M HCl(aq). The precipitate was collected by filtration and washed with a solution of water:EtOH (2:1) then water:EtOH (1:1). The resulting beige solid was dried in vacuo (100 mg, 25%). NMR; δH (400 MHz, d⁶-DMSO) 0.90 (t, 3H, J=7.5 Hz), 1.25-1.36 (m, 2H), 1.59-1.69 (m, 2H), 3.25 (s, 3H), 3.97 (t, 2H, J=7 Hz), NH not observed to δH 15; m/z 291.2[MH⁺].

### Example 2

### 1,3-dibutyl-8-(trifluoromethyl)-3,7-dihydro-1H-purine-2,6-dione

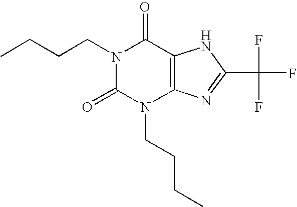

A purple mix of 5,6-diamino-1,3-dibutyl-2,4(1H,3H)-pyrimidinedione (Journal of Medicinal Chemistry (1991), 34(1), 466-9) (207 mg) and TFM (4 mm) was heated at reflux for 1 hour then concentrated in vacuo. The resulting residue was treated with 2N NaOH(aq) (5 ml) and the mixture heated at reflux for 30 minutes. After cooling to ambient temperature, the mixture was acidified to ca. pH4 with 3M HCl (aq). The precipitate was collected by filtration and purified by recrystallisation from a solution of water:EtOH (2:1). The pale brown/orange solid was dried under vacuum at 50° C. for 48 hours providing the title compound as a beige solid (35 mg, 13%). δH (400 MHz, d⁶-DMSO) 0.90 (t, 6H, J=7 Hz), 1.26-1.34 (m, 4H), 1.48-1.68 (overlapping m, 4H), 3.89 (t, 2H, J=7 Hz), 3.97 (t, 2H, J=7 Hz), NH not observed to δH 15; m/z 333.2[MH⁺]

### Example 3

### 3-butyl-1-methyl-8-(Pentafluoroethyl)-3,7-dihydro-1H-purine-2,6-dione

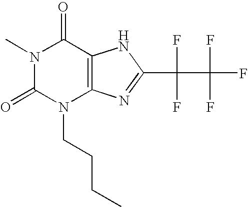

A purple mix of 5,6-diamino-1-butyl-3-methyl-2,4(1H,3H)-pyrimidinedione (European Journal of Medicinal Chemistry (1990), 25(8), 653-8) (300 mg, 1.41 mmol) and pentafluoropropionic anhydride (PFPA) (6 ml) was heated at 60° C. for 1 hour then concentrated in vacuo. The resulting residue was treated with 2N NaOH (aq) (7 ml) and the mixture heated at 90° C. for 30 minutes. After cooling to room temperature the mixture was acidified to ca. pH4 with 3M HCl(aq). The precipitate was collected by filtration, washed with water and dried under vacuum affording the title compound as a pale green/brown fluffy solid (163 mg, 34%). δH (400 MHz, d⁶-DMSO) 0.89 (t, 3H, J=7.5 Hz), 1.24-1.35 (m, 2H), 1.60-1.69 (m, 2H), 3.25 (s, 3H), 3.98 (t, 2H, J=7 Hz), NH not observed to δH 15; m/z 358.4[MNH₄⁺].

### Example 4

### 8-(difluoromethyl)-1-methyl-3-pentyl-3,7-dihydro-1H-purine-2,6-dione

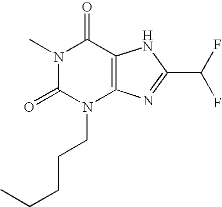

A solution of 8-(difluoromethyl)-1-methyl-3-pentyl-7-(2-propen-1-yl)-3,7-dihydro-1H-purine-2,6-dione in anhydrous THF (3 ml) and anhydrous DMSO (0.3 ml) was degassed under a gentle vacuum and nitrogen introduced. This was repeated twice. Pd(PPh₃)₄ (74 mg, 0.064 mmol) was added and the mixture degassed once more. Morpholine (374 uL, 4.3 mmol) was added and left to stir at rt for 4 hours. The mixture was partitioned between 2M HCl(aq) and EtOAc. The organic layer was separated, washed with brine, dried (MgSO₄) and concentrated. The residue was taken up in MeOH and passed down and amino-propyl SPE column (5 g), eluting with MeOH followed by 5% AcOH/MeOH. The product fraction was concentrated to afford the title compound as an off white solid (98 mg, 80%). δH (400 MHz, d⁶-DMSO) 0.85 (t, 3H, J=7 Hz), 1.23-1.35 (m, 4H), 1.61-1.71 (m, 2H), 3.25 (s, 3H), 3.96 (t, 2H, J=7.5 Hz), 7.12 (t, 1H, J=53 Hz), 14.59 (br. s, 1H); m/z 287.2[MH⁺].

### 8-(difluoromethyl)-1-methyl-3-pentyl-7-(2-propen-1-yl)-3,7-dihydro-1H-purine-2,6-dione

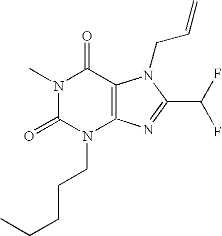

1-methyl-2,6-dioxo-3-pentyl-7-(2-propen-1-yl)-2,3,6,7-tetrahydro-1H-purine-8-carbaldehyde (133 mg, 0.44 mmol) was added to a pre-dried flask under nitrogen. Anhydrous DCM (3 ml) was added followed by deoxofluor (161 ul, 0.87 mmol) and finally EtOH (1 drop, ca. 0.09 mmol). The mixture was allowed to stir at rt. for 4 hours then partitioned between sat. NaHCO₃(aq) and DCM. The organic layer was separated, washed with brine, dried (MgSO₄) and concentrated to afford the title compound as a yellow oil (145 mg). m/z 327.4[MH⁺].

### 1-methyl-2,6-dioxo-3-pentyl-7-(2-propen-1-yl)-2,3,6,7-tetrahydro-1H-purine-8-carbaldehyde

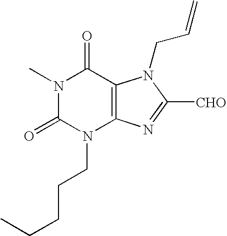

To a solution of 1-methyl-3-pentyl-7-(2-propen-1-yl)-3,7-dihydro-1H-purine-2,6-dione (1.05 g, 3.8 mmol) in THF (15 ml) at −78° C. was added lithium hexamethyldisilylazide (1M solution in hexane, 4 ml, 4 mmol) over 10 min. The solution was stirred for 0.5 h, then DMF (0.5 ml, 6.5 mmol) added, and the solution stirred for a further 0.5 h at −78° C. after which it was allowed to warm to rt over 2 h. The reaction was quenched with 2N HCl solution then separated between EtOAc/brine. Chromatography over silica (gradient elution with DCM to 5:1 DCM/EtOAc) provided the title compound (0.35 g, 30%). m/z 305[MH⁺].

### 1-methyl-3-pentyl-7-(2-propen-1-yl)-3,7-dihydro-1H-purine-2,6-dione

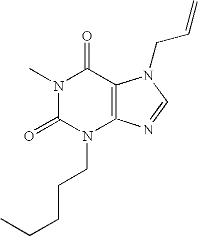

To a solution of 3-pentyl-7-(2-propen-1-yl)-3,7-dihydro-1H-purine-2,6-dione (0.20 g, 0.76 mmol) and methyl iodide (0.5 ml, 4.9 mmol) in DMF (5 ml) was added potassium carbonate (0.4 g, 2.9 mmol) and the mixture heated at 50° C. for 3 h, cooled and the mixture separated between EtOAc/brine. The organics were isolated, dried (MgSO₄) and concentrated to yield the required product (0.21 g, 100%). m/z 277[MH⁺].

### 3-pentyl-7-(2-propen-1-yl)-3,7-dihydro-1H-purine-2,6-dione

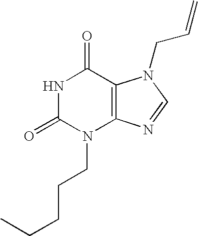

To a solution of 7-(2-propen-1-yl)-3,7-dihydro-1H-purine-2,6-dione (0.61 g, 3.2 mmol) and pentyl iodide (0.64 g, 3.2 mmol) in DMF (7 ml) was added sodium carbonate (0.60 g, 5.7 mmol) and the mixture heated at 50° C. for 18 h. After cooling and the mixture was separated between EtOAc/brine. The organics were isolated, dried (MgSO₄) and concentrated to yield the crude product which was purified by chromatography over silica (gradient elution with DCM to 5:1 DCM/EtOAc) providing the title compound (0.47 g, 56%). m/z 263[MH⁺].

### Example 5

### 3-pentyl-8-(trifluoromethyl)-3,7-dihydro-1H-purine-2,6-dione

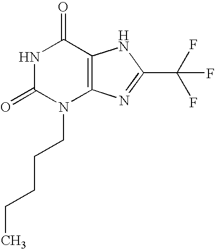

A mixture of 6-amino-5-nitroso-1-pentyl-2,4(1H,3H)-pyrimidinedione (500 mg, 2.21 mmol), 10% Pd/C (100 mg) and EtOH (100 ml) was vigorously stirred under an atmosphere of hydrogen for 4 hours. The mixture was filtered through Celite and the filtrate concentrated in vacuo, giving 5,6-diamino-1-pentyl-2,4(1H,3H)-pyrimidinedione as a pale green solid (62 mg) and as a mixture with an unknown product (2:1). Trifluoroacetic anhydride was added (5 ml) and heated at reflux for 1 hour then concentrated in vacuo. 2M NaOH(aq) (15 ml) was added to the crude residue and heated at 100° C. for 30 minutes. After allowing to cool to rt. the mixture was acidified to ca. pH 5 with 2M HCl(aq) The resulting precipitate was collected by filtration and then taken up into MeOH with heating and then passed down an amino propyl column (5 g), eluting with 2% AcOH/MeOH, 5% AcOH/MeOH, 10% AcOH/MeOH and 15% AcOH/MeOH. The product fraction was concentrated, giving the title compound as an off-white solid (52 mg). NMR; (400 MHz, d⁶-DMSO) δH 0.85 (t, 3H, J=7 Hz), 1.22-1.31 (m, 4H), 1.61-1.66 (quintet, 2H, J=7 Hz), 3.89 (t, 2H, J=7 Hz), 11.39 (s, 1H), 15.3 (br. s, 1H). m/z 291.2 [MH⁺].

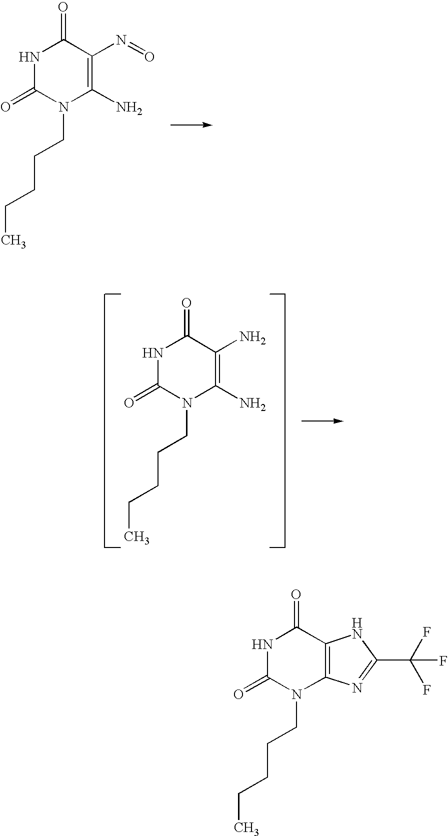

### 6-amino-5-nitroso-1-pentyl-2,4(1H,3H)-pyrimidinedione

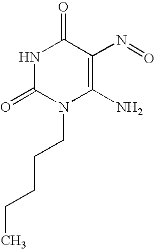

A mixture of 6-amino-1-pentyl-2,4(1H,3H)-pyrimidinedione (3.0 g, 15.2 mmol), AcOH (6 ml) and 6M HCl(aq) was cooled to 0° C. then treated dropwise with a solution of NaNO₂ (1.2 g, 16-7 mmol) in water (4 ml). The mixture was stirred for 45 minutes and water added (ca. 200 ml). The resulting purple solid was filtered, washed with water (ca. 200 ml) and dried under high vacuum at 60° C. for 18 hours. Gave the title compound as a purple solid (2.08 g, 60%). m/z 227.3 [MH⁺].

### 6-amino-1-pentyl-2,4(1H,3H)-pyrimidinedione

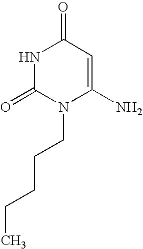

A mixture of 2-cyano-N-[(pentylamino)carbonyl]acetamide (17.9 g, 0.091 mol), EtOH (70 ml) and water (40 ml) was heated to 85° C. and then 10% NaOH(aq) (10 ml) was added dropwise. The reaction was stirred for 1 hour before allowing to cool to rt. then diluted with water and acidified to ca. pH6 using 2M HCl(aq). The mixture was cooled (ice-bath) then filtered and the resulting solid washed with water then dried under vacuum at 40° C. This gave the title compound as a white solid (11.0 g, 61%). m/z 198.2 [MH⁺].

### 2-cyano-N-[(pentylamino)carbonyl]acetamide

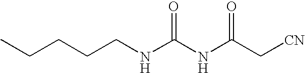

A mixture of pentyl urea (16.4 g, 0.13 mol), cyanoacetic acid (11.8 g, 0.14 mol) and acetic anhydride (30 ml) was heated at 80° C. for 2 hours. The mixture was allowed to cool to rt. and stirred with Et₂O (ca. 100 ml) for 1 hour. The mixture was cooled to 0° C. (ice-bath) and the solid collected by filtration giving the title compound as a white solid (17.9 g, 72%). m/z 196.1 [MH⁺].

### Example 5(b)

### 3-Pentyl-8-(trifluoromethyl)-3,7-dihydro-1H-purine-2,6-dione (alternative method)

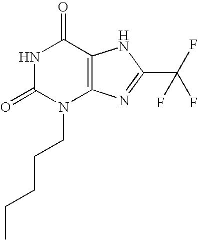

To a suspension of 6-amino-5-nitroso-1-pentyl-2,4(1H,3H)-pyrimidinedione (0.227 g, 1 mmol) in anhydrous dichloromethane (5 ml) was added trifluoroacetic anhydride (0.35 ml, 2.5 mmol).

The resulting pale yellow suspension was stirred for 40 min at room temperature and then the solution was concentrated in vacuo.

The residue was dissolved in dioxan (25 ml) and added to 10% palladium on carbon (0.5 g) and hydrogenated for 1 hr. The catalyst was filtered off, washed with dioxan and the filtrate concentrated in vacuo. Some starting material remained so the hydrogenation step was repeated. After a further 1.5 h the catalyst was filtered off, washed with dioxan and the filtrate concentrated in vacuo.

The residue was dissolved in tetrahydrofuran (10 ml) and 2N NaOH (5 ml) was added. The solution was stirred at 60° C. for 2 h. The crude reaction mixture was diluted with ethyl acetate and water and then acidified with 2N HCl. The organic phase was washed with brine, dried (MgSO₄) and concentrated in vacuo to give the title compound as an orange solid (140 mg 48%).

LC/MS: m/z 291 [MH]⁺, RT 3.00 min

¹H NMR (DMSO-d₆) δ_{H} 0.85 (3H, t, J=7 Hz), 1.28 (4H, m), 1.64 (2H, m), 3.88 (2H, t, J=7 Hz), 11.22 (1H, br s).

### Example 6

### 1-(2-hydroxyethyl)-3-pentyl-8-(trifluoromethyl)-3,7-dihydro-1H-purine-2,6-dione

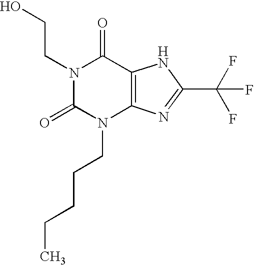

A solution of 6-amino-1-pentyl-3-[2-(tetrahydro-2H-pyran-2-yloxy)ethyl]-2,4(1H,3H)-pyrimidinedione (970 mg) in AcOH (10 ml) was treated with 6M HCl(aq) (8 ml) and then a solution of NaNO₂ (247 mg) in water (3 ml) was added. After 15 minutes the mixture was partitioned between water and EtOAc. The organic layer was separated and the aqueous layer extracted with EtOAc (×7). The combined extracts were washed with brine, dried (MgSO₄) and concentrated, giving a purple oil (446 mg). A suspension of this oil in DCM (12 ml) was treated with the dropwise addition of TFAA (12 ml). After 10 minutes the mixture was concentrated giving a yellow/brown oil (798 mg). A portion of this material (150 mg) was reacted under an atmosphere of hydrogen in THF (15 ml) for 2.5 hours. The mixture was filtered through celite and washed through with THF. The filtrate was concentrated and treated once more with TFAA (5 ml) and then concentrated in vacuo. 2M NaOH(aq) (4 ml) was added to the residue and the mixture heated at 50° C. for 30 minutes. After cooling, the mixture was acidified to ca. pH 5 and the product extracted with EtOAc. The organic layer was separated, washed with brine, dried (MgSO₄) and concentrated. The resulting residue was passed down an auotoprep hplc to furnish the title compound (32 mg, 17%). NMR; (400 MHz, d6-DMSO) δH 0.85 (t, 3H, J=7 Hz), 1.23-1.36 (m, 4H), 1.62-1.70 (m, 2H), 3.52 (t, 2H, J=6.5 Hz), 3.97 (m, 4H), 15.5 (br. S, 1H). m/z 335.3 [MH+].

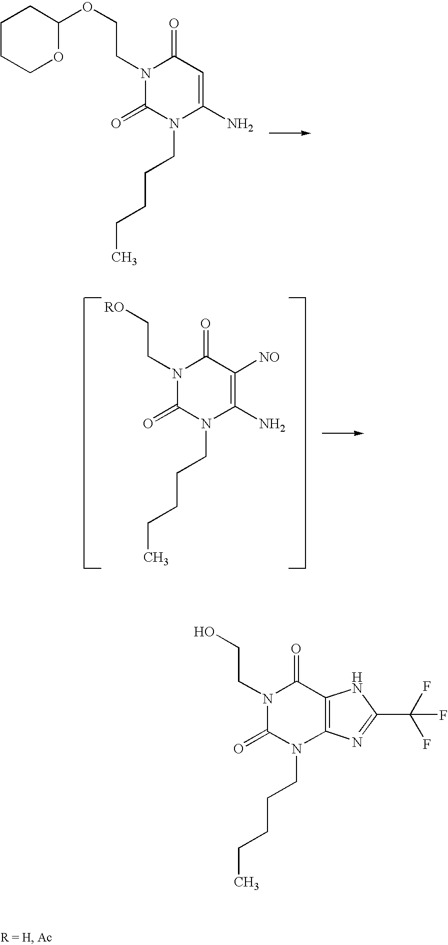

### 6-amino-1-pentyl-3-[2-(tetrahydro-2H-pyran-2-yloxy)ethyl]-2,4(1H,3H)-pyrimidinedione

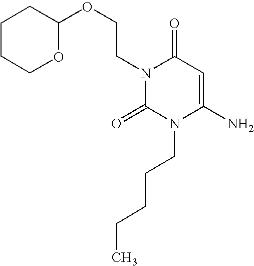

A mixture of 6-amino-1-pentyl-2,4(1H,3H)-pyrimidinedione (300 mg, 1.5 mmol), Cs₂CO₃ (595 mg, 1.8 mmol), 2-(2-bromoethoxy)tetrahydro-2H-pyran (276 ul, 1.8 mmol) and anhydrous DMF (5 ml) was heated at 40° C. for 18 hours. The mixture was partitioned between water and EtOAc. The organic layer was separated, washed with brine, dried (MgSO₄) and concentrated, giving a pale brown gum (198 mg, 40%). m/z 326.3 [MH⁺].

### Example 7(a)

### 3-Butyl-1-{3-[3-(phenylmethyl)-1,2,4-oxadiazol-5-yl]propyl}-8-(trifluoromethyl)-3,7-dihydro-1H-purine-2,6-dione

### Example 7(b)

### 4-[3-Butyl-2,6-dioxo-8-(trifluoromethyl)-2,3,6,7-tetrahydro-1H-purin-1-yl]butanoic acid

### a) 3-Butyl-1-{3-[3-(phenyl methyl)-1,2,4-oxadiazol-5-yl]propyl}-8-(trifluoromethyl)-3,7-dihydro-1H-purine-2,6-dione

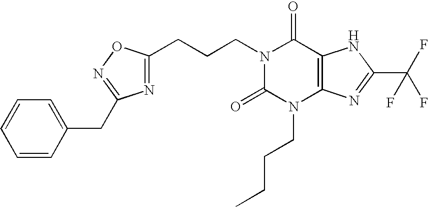

4-[3-Butyl-2,6-dioxo-8-(trifluoromethyl)-2,3,6,7-tetrahydro-1H-purin-1-yl]butanoic acid (0.181 g, 0.5 mmol) and CDI (0.089 g, 0.55 mmol) were dissolved in anhydrous dimethylformamide (6 ml). After stirring the solution under nitrogen for 3 h at room temperature, benzylamidoxime (0.083 g, 0.55 mmol) was added and the solution heated at 90° C. for 20 h, then at 110° C. for 4 h. The reaction mixture was concentrated in vacuo to a yellow oil. This was purified on a Teledyne Isco CombiFlash® Companion® using a 40 g RediSep silica cartridge (gradient elution with chloroform/ethanol 0-15%). The appropriate fractions were evaporated in vacuo, than freeze dried from dioxan to give the title compound as a white lyophilate (142 mg, 59%)

LC/MS: m/z 477 [MH]⁺, RT 3.57 min

¹H NMR (CDCl₃) δ_{H} 0.98 (3H, t, J=7 Hz), 1.41 (2H, m), 1.76 (2H, m), 2.23 (2H, m), 2.95 (2H, t, J=8 Hz), 4.00 (2H, s), 4.14 (2H, t, J=8 Hz), 4.22 (2H, t, J=7 Hz), 7.27 (5H, s)

### b) 4-[3-Butyl-2,6-dioxo-8-(trifluoromethyl)-2,3,6,7-tetrahydro-1H-purin-1-yl]butanoic acid

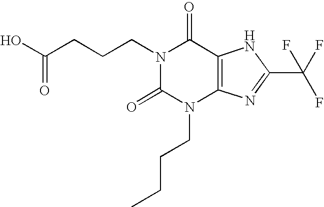

Anhydrous dichloromethane (50 ml) was added to ethyl 4-(4-amino-3-butyl-5-nitroso-2,6-dioxo-3,6-dihydro-1(2H)-pyrimidinyl)butanoate (3.26 g, 10 mmol), followed by trifluoroacetic anhydride (3.53 ml, 25 mmol). The yellow solution was stirred at room temperature under an atmosphere of nitrogen for 15 min and then concentrated in vacuo. The residue was dissolved in dioxan (150 ml) and added to 10% palladium on carbon (0.5 g) and hydrogenated for 2 h. The catalyst was filtered off, washed with dioxan and the filtrate concentrated in vacuo.

The residue was dissolved in tetrahydrofuran (100 ml) and 2N NaOH (50 ml) was added. The solution was stirred at 60° C. for 1.5 h cooled and diluted with ethyl acetate (100 ml) and water (30 ml). The organic phase was extracted with water (2×30 ml). The combined aqueous extracts were acidified to pH4 with 2N HCl. The resulting solid was filtered and washed with water to give the title compound as pale cream solid 2.17 g (60%).

LC/MS: m/z 363 [MH]⁺, RT 2.84 min

¹H NMR (DMSO-d₆) δ_{H} 0.90 (3H, t, J=7 Hz), 1.31 (2H, m), 1.64 (2H, m), 1.80 (2H, m), 2.24 (2H, t, J=7 Hz), 3.89 (2H, t, J=7 Hz), 3.95 (4H, m), 12.01, (1H, br s), 15.36, (1H, br s)

### c) Ethyl 4-(4-amino-3-butyl-5-nitroso-2,6-dioxo-3,6-dihydro-1(2H)-pyrimidinyl)butanoate

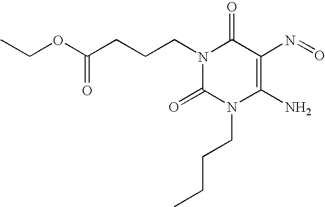

Ethyl 4-(4-amino-3-butyl-2,6-dioxo-3,6-dihydro-1(2H)-pyrimidinyl)butanoate (8.5 g, 29.8 mmol) was dissolved in a mixture of glacial acetic acid (45 ml) and 6M hydrochloric acid (24 ml). The solution was cooled to −1° C. and sodium nitrite (2.26 g, 32.7 mmol) in water (20 ml) was added dropwise. After the addition was complete the ice bath was removed and the purple solution stirred for 1 h. The solution was diluted with water (140 ml) and the product, which separated out after a few minutes, was filtered, washed with water (total 350 ml) and dried in vacuo at 50° C. to give the title compound as a bright pink solid (6.61 g). On standing a further crop was isolated (0.53 g), then the mother liquors were basified to pH6 with 2N sodium hydroxide and extracted into ethyl acetate (2×250 mL). The combined extracts were concentrated in vacuo to give a third crop of the title compound (1.23 g). Total yield 8.37 g 86%

LC/MS: m/z 327 [MH]⁺, RT 2.58 min

### d) Ethyl 4-(4-amino-3-butyl-2,6-dioxo-3,6-dihydro-1(2H)-pyrimidinyl)butanoate

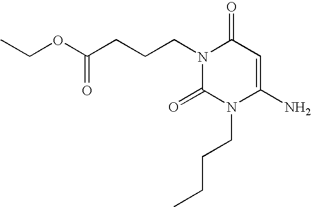

6-Amino-1-butyl-2,4(1H,3H)-pyrimidinedione (5.9 g, 32.2 mmol) and caesium carbonate (11.54 g, 35.4 mmol) were added to anhydrous dimethylformamide under a nitrogen atmosphere. Ethyl 4-bromobutyrate (5.07 ml, 35.4 mmol) was added and the mixture stirred at room temperature for 22 h. The reaction mixture was diluted with ethyl acetate (350 ml) and washed with saturated sodium chloride solution (2×50 ml). The combined aqueous phases were back extracted with ethyl acetate. The combined organic phases were washed with saturated sodium chloride solution, dried (MgSO₄) and concentrated in vacuo. The crude product was purified on a Teledyne Isco CombiFlash) Companions using a 80 g RediSep silica cartridge (gradient elution using chloroform/ethanol 0-40%). The appropriate fractions were evaporated in vacuo to give the title compound as a yellow oil which crystallised on standing (5.36 g, 56%)

LC/MS: m/z 298 [MH]⁺, RT 2.49 min

### Example 8(a)

### 3-Butyl-1-[4-(3-phenyl-1,2,4-oxadiazol-5-yl)butyl]-8-(trifluoromethyl)-3,7-dihydro-1H-purine-2,6-dione

### Example 8(b)

### 5-[3-butyl-2,6-dioxo-8-(trifluoromethyl)-2,3,6,7-tetrahydro-1H-purin-1-yl]pentanoic acid

### a) 3-Butyl-1-[4-(3-phenyl-1,2,4-oxadiazol-5-yl)butyl]-8-(trifluoromethyl)-3,7-dihydro-1H-purine-2,6-dione

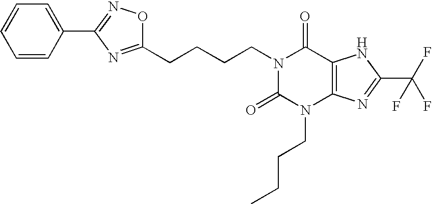

A mixture of methyl 5-[3-butyl-2,6-dioxo-8-(trifluoromethyl)-2,3,6,7-tetrahydro-1H-purin-1-yl]pentanoate (0.156 g, 0.4 mmol), benzamidoxime (60 mg, 0.44 mmol), 21% solution of NaOEt in EtOH (0.22 ml, 0.6 mmol) and anhydrous EtOH (1 ml) was heated in the microwave at 140° C. for 10 min. The mixture was diluted with EtOAc (30 ml) and washed with water (20 ml), 2N HCl (20 ml) and brine (5 ml). The organic layer was dried (Na₂SO₄) and concentrated in vacuo to a yellow oil and then freeze dried from dioxan to give the title compound as a cream lyophilate (95 mg, 50%)

LC/MS: m/z 476 [MH]⁺, RT 3.73 min

¹H NMR (CDCl₃) δ_{M} 0.97 (3H, t, J=7 Hz), 1.41 (2H, m), 1.77 (2H, m), 1.87 (2H, m), 1.97 (2H, m), 3.02 (2H, t, J=7 Hz), 4.17 (4H, m), 7.47 (3H, m), 8.04 (2H, m)

### b) 5-[3-butyl-2,6-dioxo-8-(trifluoromethyl)-2,3,6,7-tetrahydro-1H-purin-1-yl]pentanoic acid

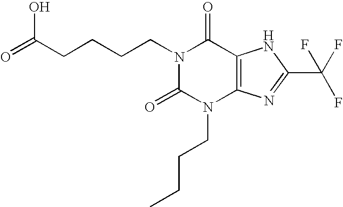

and methyl 5-[3-butyl-2,6-dioxo-8-(trifluoromethyl)-2,3,6,7-tetrahydro-1H-purin-1-yl]pentanoate

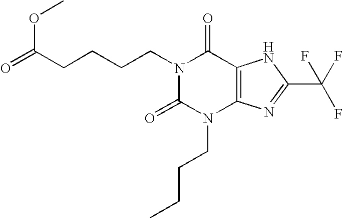

Anhydrous dichloromethane (70 ml) was added to methyl 4-(4-amino-3-butyl-5-nitroso-2,6-dioxo-3,6-dihydro-1(2H)-pyrimidinyl)pentanoate (5.06 g, 15.5 mmol), followed by trifluoroacetic anhydride (22 ml, 10 equiv.). The yellow solution was stirred at room temperature under an atmosphere of nitrogen for 25 min and then concentrated in vacuo.

The residue was dissolved in dioxan (250 ml) and added to 10% palladium on carbon (0.85 g) and hydrogenated for 2.25 h. The catalyst was filtered off, washed with dioxan and the filtrate concentrated in vacuo. The residue was dissolved in tetrahydrofuran (100 ml) and 2N NaOH (15.5 ml) was added. The solution was stirred at room temperature for 3.5 h. A further 8 ml of 2N NaOH was added and the reaction mixture heated at 60° C. for 1.5 h. The reaction mixture was cooled and concentrated in vacuo to a small volume and partitioned between ethyl acetate and water. The organic phase was washed with water (2×) and brine. The combined aqueous extracts were acidified to with 2N HCl, extracted into ethyl acetate (5×40 ml) and then back extracted with saturated NaHCO₃ (3×30 ml) and brine (30 ml). The combined aqueous phases were acidified to pH 4.5 with 2N HCl and resulting solid was filtered and washed with water to give 5-[3-butyl-2,6-dioxo-8-(trifluoromethyl)-2,3,6,7-tetrahydro-1H-purin-1-yl]pentanoic acid as a cream solid 2.19 g (38%).

The organic phase was dried over MgSO₄ and concentrated in vacuo to a yellow gum which solidified on standing to give methyl 5-[3-butyl-2,6-dioxo-8-(trifluoromethyl)-2,3,6,7-tetrahydro-1H-purin-1-yl]pentanoate as a peach coloured solid (0.9 g, 15%)

Data for 5-[3-butyl-2,6-dioxo-8-(trifluoromethyl)-2,3,6,7-tetrahydro-1H-purin-1-yl]pentanoic acid:

LC/MS: m/z 377 [MH]⁺, RT 2.91 min

¹H NMR (DMSO-d₆) δ_{H} 0.90 (3H, t, J=7 Hz), 1.30 (2H, t, J=7 Hz), 1.45-1.68 (6H, m), 2.24 (2H, t, J=7 Hz), 3.89 (2H, t, J=7 Hz), 3.97 (2H, t, J=7 Hz), 12.00 (1H, br s)

Data for methyl 5-[3-butyl-2,6-dioxa-8-(trifluoromethyl)-2,3,6,7-tetrahydro-1H-purin-1-yl]pentanoate:

LC/MS: m/z 391 [MH]⁺, RT 3.15 min.

¹H NMR (CDCl₃) δ_{H} 0.95 (3H, t, J=7 Hz), 1.27 (2H, t, J=7 Hz), 1.38 (2H, m) 1.75 (2H, m), 2.06 (2H, s), 2.36 (2H, t, J=7 Hz), 3.62 (3H, s), 4.03 (2H, br), 4.14 (2H, m)

### c) Methyl 4-(4-amino-3-butyl-5-nitroso-2,6-dioxo-3,6-dihydro-1(2H)-pyrimidinyl)pentanoate

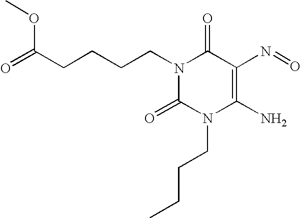

Prepared in similar fashion to ethyl 4-(4-amino-3-butyl-5-nitroso-2,6-dioxo-3,6-dihydro-1(2H)-pyrimidinyl)butanoate

LC/MS: m/z 327 [MH]⁺, RT 2.54 min

### d) Methyl 5-(4-amino-3-butyl-2,6-dioxo-3,6-dihydro-1(2H)pyrimidinyl)pentanoate

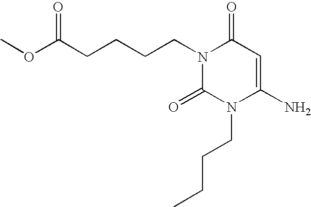

Prepared in the same way as ethyl 4-(4-amino-3-butyl-2,6-dioxo-3,6-dihydro-1(2H)-pyrimidinyl)butanoate

LC/MS: m/z 298 [MH]⁺, RT 2.47 min

### Example 9

### 3-Butyl-{4-[3-(2-fluorophenyl)-1,2,4-oxadiazol-5-yl]butyl}-8-(trifluoromethyl-3,7-dihydro-1H-purine-2,6-dione

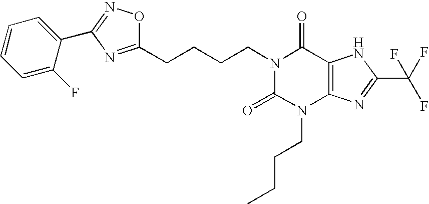

5-[3-butyl-2,6-dioxo-8-(trifluoromethyl)-2,3,6,7-tetrahydro-1H-purin-1-yl]pentanoic acid (0.301 g, 0.8 mmol) and CDI (0.153 g, 0.94 mmol) were dissolved in anhydrous dimethylformamide (10 ml). After stirring the solution under nitrogen for 4 h at room temperature, 2-fluorobenzamidoxime (0.135 g, 0.88 mmol) was added and the solution heated at 85° C. for 19 h. The reaction mixture was concentrated in vacuo and purified an a 10 g aminopropyl SPE (10 g) eluting with methanol. Evaporation of the methanol eluant gave an oil that was purified further by MDAP. The relevant fractions were combined and concentrated in vacuo and the residue was freeze dried from dioxan to give the title compound (0.037 g, 9%) as a white lyophilate.LC/MS: m/z 495 [MH], RT 3.68 min

¹H NMR (CDCl₃) δ_{H} 0.97 (3H, t, J=7 Hz), 1.40 (2H, m), 1.77 (2H, m), 1.88 (2H, m), 1.97 (2H, m), 3.05.(2H, t, J=7 Hz), 4.17 (2H, q, J=7 Hz), 7.19-7.29 (2H, m) 7.45-7.48 (1H, m) 8_{—}02, (1H, dt, J=8 and 2 Hz)

### Example 10

### 3-Butyl-1-{4-[3-(4-fluorophenyl)-1,2,4-oxadiazol-5-yl]butyl}-8-(trifluoromethyl)-3,7-dihydro-1H-purine-2,6-dione

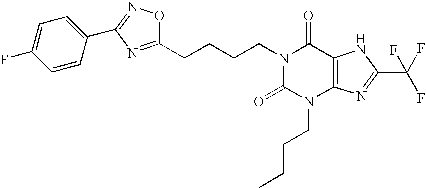

5-[3-butyl-2,6-dioxo-8-(trifluoromethyl)-2,3,6,7-tetrahydro-1H-purin-1-yl]pentanoic acid (0.301 g, 0.8 mmol) and CDI (0.153 g, 0.94 mmol) were dissolved in anhydrous dimethylformamide (10 ml). After stirring the solution under nitrogen for 4 h at room temperature 4-fluorobenzamidoxime (0.135 g, 0.88 mmol) was added and the solution heated at 85° C. for 22 h. The reaction mixture was concentrated in vacuo, taken up in ethyl acetate and washed with brine (2×20 ml) and 0.1N HCl. The organic phase was dried (MgSO₄) and concentrated in vacuo to give the required product as a pale yellow oil which crystallised on standing. This material was freeze dried from dioxan to give the title compound as a cream lyophilate (0.225 g, 57%).

LC/MS: m/z 495 [MH]⁺, RT 3.77 min

¹H NMR (CDCl₃) δ_{H} 0.97 (3H, t, J=7 Hz), 1.27 (2H, m), 1.77 (2H, m), 1.87 (2H, m,), 1.97 (2H, m), 3.02 (2H, t, J=7 Hz), 4.17 (4H, m,), 7.15 (2H, m, J=9 Hz), 8.04 (2H, m).

### Example 11

### 3-Butyl-1-{4-[3-(2-pyridinyl)-1,2,4-oxadiazol-5-yl]butyl}-8-(trifluoromethyl)-3,7-dihydro-1H-purine-2,6-dione

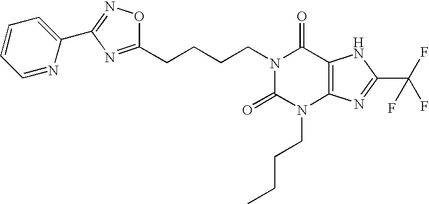

5-[3-butyl-2,6-dioxo-8-(trifluoromethyl)-2,3,6,7-tetrahydro-1H-purin-1-yl]pentanoic acid (0.15 g, 0.4 mmol) and CDI (0.078 g, 0.48 mmol) were dissolved in anhydrous dimethylformamide (5 ml). After stirring the solution under nitrogen for 1.5 h at room temperature 2-pyridylamidoxime (0.06 g, 0.44 mmol) was added and the solution heated at 90° C. for 16.5 h. The reaction mixture was concentrated in vacuo and purified by MDAP. The relevant fractions were evaporated to give the title compound as a tan solid (0.017 g, 9%).

LC/MS: m/z 478 [MH]⁺, RT 3.28 min

¹H NMR (CDCl₃) δ_{H} 0.95 (3H, t, J=7 Hz), 1.39 (2H, m, J=7 Hz), 1.75 (2H, m), 1.84 (2H, m), 1.96 (2H, m), 3.07 (2H, t, J=7 Hz), 4.14 (4H, m), 7.41 (1H, m) 7.84 (1H, dt, J=8 and 2 Hz), 8.10 (1H, d, J=8 Hz), 8.75 (1H, d, J=4 Hz)

### Example 12

### 3-Butyl-8-(trifluoromethyl)-1-(3-{3-[(2,4,6-trifluorophenyl)methyl]-1,2,4-oxadiazol-5-yl}propyl)-3,7-dihydro-1H-purine-2,6-dione

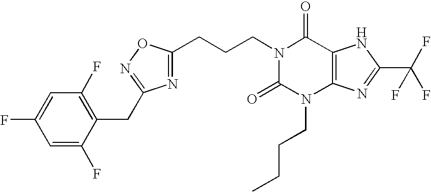

4-[3-Butyl-2,6-dioxo-8-(trifluoromethyl)-2,3,6,7-tetrahydro-1H-purin-1-yl]butanoic acid (0.181 g, 0.5 mmol) and CDI (0.089 g, 0.55 mmol) were dissolved in anhydrous dimethylformamide (6 ml). After stirring the solution under nitrogen for 3 h at room temperature. 2,4,6-trifluorobenzylamidoxime (0.112 g, 0.55 mmol) was added and the solution heated at 90° C. for 20 h, then at 110° C. for 6 h. The reaction mixture was concentrated in vacuo to a yellow oil. This was purified on a Teledyne Isco CombiFlash® Companion® using a 35 g RediSep silica cartridge (gradient elution chloroform/ethanol 0-15%). The appropriate fractions were evaporated in vacuo to give after freeze drying from dioxan the title compound as a white solid (0.122 g, 46%).

LC/MS: m/z 531 [MH]⁺, RT 3.61 min

¹H NMR (CDCl₃) δ_{H} 0.97 (3H, t, J=7 Hz), 1.41 (2H, m), 1.76 (2H, m), 2.23 (2H, m), 2.95 (2H, t, J=8 Hz), 4.00 (2H, s), 4.14 (2H, t, J=8 Hz), 4.22 (2H, t, J=7 Hz), 6.67 (2H, t, J=7 Hz).

### Example 13

### 1-Methyl-3-pentyl-8-(trifluoromethyl)-3,7-dihydro-1H-purine-2,6-dione

### a) 1-Methyl-3-pentyl-8-(trifluoromethyl)-3,7-dihydro-1H-purine-2,6-dione

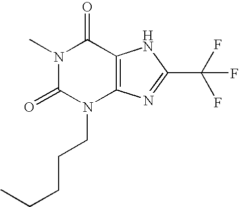

A solution of 6-amino-3-methyl-5-nitroso-1-pentyl-2,4(1H,3H)-pyrimidinedione (0.042 g, 0.18 mmol) in anhydrous DCM (2 ml) was treated with trifluoroacetic anhydride (1 ml) and left to stir at rt. for 10 mins. The solution was concentrated in vacuo and the residue taken up into 1,4-dioxane (15 ml) and reacted with 10% Pd/C (70 mg) and trifluoroacetic anhydride ((3.1 ml) under an atmosphere of hydrogen for 6 h. The mixture was filtered through celite and the filtrate concentrated, giving a red oil. THF (2 ml) was added followed by 2 KV NaOH(aq) solution. The mixture was heated to 50° C. for 10 mins. After cooling, the mixture was partitioned between 2M HCl(aq) and EtOAc. The organic layer was separated, washed with brine, dried (MgSO₄) and concentrated. The title product was purified by MDAP to give the title compound as a white solid (10 mg, 19%).

LC/MS m/z 305 [MH⁺], RT 3.2 mins.

¹H NMR; (d⁶-DMSO) δ_{H} 0.92 (3H, t, J=7 Hz), 1.38 (4H, m), 1.80 (2H, m), 3.51 (3H, s), 4.17 (2H, t, J=8 Hz), 13.5 (1H, br s).

### b) 6-amino-3-methyl-5-nitroso-1-pentyl-2,4(1H,3H)-pyrimidinedione

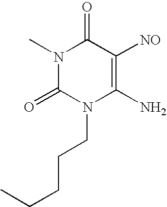

Prepared in similar fashion to ethyl 4-(4-amino-3-butyl-5-nitroso-2,6-dioxo-3,6-dihydro-1(2H)-pyrimidinyl)butanoate.

All publications, including but not limited to patents and patent applications, cited in this specification are herein incorporated by reference as if each individual publication were specifically and individually indicated to be incorporated by reference herein as though fully set forth.

1-16. (canceled)

17. A compound of formula (I): 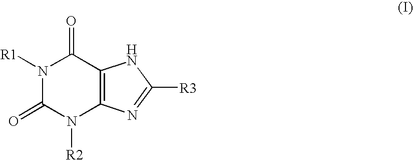
and pharmaceutically acceptable derivatives thereof
wherein:
R¹ is a group selected from hydrogen, C₁₋₁₀ alkyl, C₂₋₁₀ alkenyl, C₂₋₁₀ alkynyl and -(alk)ₘ-X-(alk)ₙ-Y,
wherein X is A, A1, A2 or a direct link;
wherein:
A is selected from cycloalkylene, cycloalkenylene, aryl, heteroaryl, heterocyclyl, —CH₂—OC(O)—;
A1 is selected from —CH₂—O—(CH₂)_{q}aryl-O—, —CH₂—O—(CH₂)_{w}N(R⁵)C(O)O—, —CH₂—N(R⁵)C(O)O—, —CH₂—N(R⁵)C(O)—, —CH₂—(O)ₚ—(CH₂)_{q}C(O)NR⁵—, —CH₂—N(R⁵)C(O)N(R⁵)—, —CH₂—C(O)N((CH₂)_{w}OH)—, —CH₂—NR⁵—S(O)₂—, CH₂—S(O)₂NR⁵—, —CH₂—C(O)O—, —O—, —NR⁵— or —S—;
A2 is —CH(OH)—;
when X is A, A1 or A2, Y is a group selected from heteroaryl, heterocyclyl, aryl, cycloalkyl, cycloalkenyl, —O(CH₂)ₙ-aryl, —C(O)O-aryl, —CH(aryl)₂, —CH(heteroaryl)₂, —C₁₋₆ haloalkyl, —C(O)R^{4,}, —NR⁵R⁷, —C(O)NR⁵R⁷, —NR⁵C(O)R⁷, —NR⁵C(O)OR⁷, —C(O)(CH₂)_{q}OR⁴, halogen, cyano, —N(R⁵)C(O)OR⁷, —OC(O)NR⁵R⁶, —NR⁵C(O)R⁸, —OR⁵, or —OC(O)R⁴;
when X is A1 and Y is selected from —O(CH₂)ₙ-aryl, —O-heteroaryl, —OR⁵, —OC(O)R⁵, —NH-aryl, —OC(O)NR⁵R⁶ and n is an integer selected from 2, 3, 4 and 5;
when X is A1 and Y is —CF₃, or when X is A2, n is an integer selected from 1, 2, 3, 4 and 5;
when X is a direct link, Y is selected from —C(O)(CH₂)_{q}OR⁵, —C(O)-aryl, —C(O)-heteroaryl, —C(O)-heterocyclyl, -heteroaryl, -heterocyclyl, -aryl, -cycloalkyl, -cycloalkenyl, —C₁₋₆ haloalkyl, -halo, -cyano, —CH(aryl)₂, —CH(heteroaryl)₂, —OR⁵, —NR⁵R⁷, —NCOOR⁸, —(O)ₚC(O)NR⁵R⁶, —NR⁵C(O)R⁸, —OR⁵, —(O)ₚC(O)R⁴ or a 3 or 4 ring fused system;
wherein the 3 or 4 ring fused system is a fused 12-18 membered tricyclic or tetracyclic ring containing 1 to 4 nitrogen atoms and wherein at least one ring is an aromatic ring;
wherein 3 or 4 fused system ring is substituted by one or more of C₁₋₆ alkyl, C₂₋₆ alkenyl, C₂₋₆ alkynyl, halogen, —NH₂, (CH₂)_{q}—NR⁵R⁷, (CH₂)_{q}(O)ₚ—(CH₂)_{q}—N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, (CH₂)_{q}—N(R⁵)—S(O)₂R³, —CH₂—S(O)₂N(R⁵)R⁶, —C₁₋₆ haloalkyl, —OCF₃, —OCH(F)₂, —OCH₂F, —COOR⁵, —OR⁵, —(R³)ₚCN, —S(O₂)R⁹, —(CH₂)ₙheteroaryl, —(CH₂)ₙheterocycyl, —(CH₂)ₙcycloalkyl, —(CH₂)ₙcycloalkenyl or —(CH₂)ₙaryl;
R² is hydrogen; or C₁₋₁₀ alkyl, C₂₋₁₀ alkenyl, C₂₋₁₀ alkynyl, cycloalkyl, cycloalkenyl, heterocyclyl, aryl, and heteroaryl, each of which is optionally substituted by one or more of: C₁₋₁₀ alkyl, C₂₋₁₀ alkenyl, C₂₋₁₀ alkynyl, cycloalkyl, heterocyclyl, aryl, heteroaryl, C₁₋₆ haloalkyl, halogen, —CN, —OR⁴, —(CH₂)ₙCOR⁴, —C(O)OR⁴, —OCOR⁴, —(CH₂)ₙNR⁵R⁵, —(NH)ₚCONR⁵R⁵, OCONR⁵R⁷, and —NHC(O)OR⁷;
R³ is fluorinated C₁₋₆ alkyl;
R⁴ is selected from: hydrogen, C₁₋₆ alkyl, C₂₋₆ alkenyl, C₂₋₆ alkynyl, —(CH₂)ₙ cycloalkyl, —(CH₂)ₙ cycloalkenyl, —(CH₂)ₙ heterocyclyl, —(CH₂)ₙ aryl, and —(CH₂)ₙ heteroaryl;
R⁵ and R⁶ are hydrogen and C₁₋₄ alkyl;
R⁷ is hydrogen, C₁₋₆ alkyl, C₂₋₆ alkenyl, C₂₋₆ alkynyl, —(CH₂)ₜ cycloalkyl, —(CH₂)ₙ cycloalkenyl, —(CH₂)ₜ heterocyclyl, —(CH₂)ₜ aryl, and —(CH₂)ₜ heteroaryl;
R⁸ is selected from C₁₋₄ alkyl;
R⁹ is selected from C₁₋₆ alkyl, C₂₋₆ alkenyl, C₂₋₆ alkynyl, —(CH₂)ₙ cycloalkyl, —(CH₂)ₙcycloalkenyl, —(CH₂)ₙheterocyclyl, —(CH₂)ₙ aryl, and —(CH₂)ₙheteroaryl, CN;
m represents an integer selected from: 0, 1, 2, 3, 4 and 5;
n represents an integer selected from: 0, 1, 2, 3, 4 and 5;
p represents an integer selected from: 0 and 1;
q represents an integer selected from: 0, 1 and 2;
t represents an integer selected from: 1 and 2;
w represents an integer selected from: 2, 3 and 4; and
provided that when:
(i) R¹ represents hydrogen or C₁₋₃ alkyl, R² is different to R¹;
(ii) R¹ represents ethyl and R³ represents CF₃, R² is other than CH₂—CH═CH₂; and
(iii) R¹ represents methyl and R³ represents CF₃, R² is other than i-butyl.

18. The compound according to claim 17, wherein R¹ is hydrogen C₁₋₁₀ alkyl, and -(alk)ₘ-X-(alk)ₙ-Y.

19. The compound according to claim 17, wherein X represents A or a direct link.

20. The compound according to claim 17, wherein R¹ is selected from: hydrogen and C₁₋₆ alkyl.

21. The compound according to claim 17, wherein R² is selected from: C₄₋₆ alkyl.

22. The compound according to claim 17, wherein R³ represents CF₃.

23. A pharmaceutical formulation, which comprises at least one compound of formula (I) according to claim 17 and one or more pharmaceutically acceptable diluents, excipients or carriers.

24. A pharmaceutical combination administered together or separately, sequentially or simultaneously in separate or combined pharmaceutical formulations., wherein the pharmaceutical combination comprises at least one chemical entity according to claim 17 together with another therapeutically active agent.

25. A pharmaceutical formulation comprising:
(i) At least one chemical entity according to claim 17;
(ii) one or more active ingredients selected from statins, fibrates, bile-acid binding resins and nicotinic acid; and
(iii) one or more pharmaceutically acceptable diluents, excipients or carriers.

26. A method for treatment of a disorder of lipid metabolism or diabetes, which comprises administering to a human or an animal subject a therapeutically effective amount of a compound of formula (I) according to claim 17.

27. The method according to claim 26, wherein the disorder of lipid metabolism or diabetes is selected from dyslipidaemia, hyperlipoproteinaemia, didiabetic dyslipidaemia, mixed dyslipidaemia, hypercholesteraemia, type II diabetes mellitus, type I diabetes, obesity, insulin resistance or hyperlipidaemia.

28. A method for treatment of a cardiovascular disease or an inflammatory disease, which comprises administering to a human or an animal subject a therapeutically effective amount of a compound of formula (I) according to claim 17.

29. The method for treatment of cardiovascular disease according to claim 28, wherein the cardiovascular disease is selected from atherosclerosis, arteriosclerosis, hypertriglyceridaemia, coronary artery disease, thrombosis, angina, chronic renal failure, heart failure, peripheral vascular disease or stroke.

30. A method for treatment of anorexia nervosa, which comprises administering to a human or an animal subject a therapeutically effective amount of a compound of formula (I) according to claim 17.
